(19) United States
(12) Patent Application Publication
(10) Pub. No.: US 2025/0295330 A1
(43) Pub. Date: Sep. 25, 2025

(54) DETERMINATION DEVICE, DETERMINATION METHOD, AND RECORDING

(71) Applicant: NEC Corporation, Tokyo (JP)

(72) Inventor: Hiroshi KAJITANI, Tokyo (JP)

(73) Assignee: NEC Corporation, Tokyo (JP)

(21) Appl. No.: 18/967,842

(22) Filed: Dec. 4, 2024

(30) Foreign Application Priority Data
Mar. 25, 2024 (JP) 2024-047500

(51) Int. Cl. A61B 5/11; A61B 5/00

(52) U.S. Cl. CPC A61B 5/112; A61B 5/6807; A61B 5/7267; A61B 5/742; A61B 2562/0219

(57) ABSTRACT

Provided is a determination device including a data acquisition unit that acquires sensor data measured in accordance with a motion of a foot, a calculation unit that calculates a relative change value indicating a relative change from a traveling axis for each gait cycle using time-series data of the acquired sensor data, a determination unit that determines a gait situation using time-series data of the relative change value in a target period, and an output unit that outputs gait information including the determined gait situation.

This application is based upon and claims the benefit of priority from Japanese Patent Application No. 2024-047500, filed on Mar. 25, 2024, the disclosure of which is incorporated herein in its entirety by reference.

### TECHNICAL FIELD

The present disclosure relates to a determination device, a determination method, and a recording medium.

### BACKGROUND ART

With growing interest in healthcare, services that provide information according to a gait have attracted attention. For example, a technique for analyzing a gait using sensor data measured by a sensor mounted in footwear such as shoes has been developed. A feature associated with a gait event related to a physical condition appears in the time-series data of the sensor data. When the health condition of the subject can be estimated by the feature associated with the gait event, early detection and prevention of diseases can be performed.

PTL 1 (JP 2019-217182 A) discloses a gait state measurement device in which a measuring unit that acquires gait information is used as an insole of a shoe. The device of PTL 1 acquires acceleration data of a foot in a vertical direction and elevation angle data of a toe of the foot.

To accurately estimate the health condition of the subject, it is preferable to use data measured in a linear gait (normal gait) on a flat land. However, in the method of PTL 1, a gait (exceptional gait) on a meandering road, stair, or slope cannot be distinguished from a normal gait. Therefore, in the method of PTL 1, there is a possibility that it is determined that there is an abnormality in the health state of the subject based on the data measured in the exceptional gait. In the case of a subject having no health problem, data measured in the exceptional gait can be removed by setting a threshold value to the data. However, for a subject having a decline in muscle strength such as an elderly person or a rehabilitation patient, the strength of data is weak, and it has been difficult to distinguish between an exceptional gait and a normal gait. Even for an any subject including an elderly person, a rehabilitation patient, and the like, it is required that the gait situation can be determined using data related to gait.

An object of the present disclosure is to provide a determination device, a determination method, and a program capable of determines a gait situation of an any subject.

### SUMMARY

A determination device according to an aspect of the present disclosure includes a data acquisition unit that acquires sensor data measured in accordance with a motion of a foot, a calculation unit that calculates a relative change value indicating a relative change from a traveling axis for each gait cycle using time-series data of the acquired sensor data, a determination unit that determines a gait situation using time-series data of the relative change value in a target period, and an output unit that outputs gait information including the determined gait situation.

A determination method according to an aspect of the present disclosure includes acquiring sensor data measured in accordance with a motion of a foot, calculating a relative change value indicating a relative change from a traveling axis for each gait cycle using time-series data of the acquired sensor data, determining a gait situation using time-series data of the relative change value in a target period, and outputting gait information including the determined gait situation.

A program according to an aspect of the present disclosure causes a computer to execute the steps of acquiring sensor data measured in accordance with a motion of a foot, calculating a relative change value indicating a relative change from a traveling axis for each gait cycle using time-series data of the acquired sensor data, determining a gait situation using time-series data of the relative change value in a target period, and outputting gait information including the determined gait situation.

### BRIEF DESCRIPTION OF THE DRAWINGS

Exemplary features and advantages of the present invention will become apparent from the following detailed description when taken with the accompanying drawings in which:

FIG. 1 is a block diagram illustrating an example of a configuration of a gait measurement system in the present disclosure;

FIG. 2 is a block diagram illustrating an example of a configuration of a measurement device in the present disclosure;

FIG. 3 is a conceptual diagram illustrating an example in which the measurement device in the present disclosure is disposed in shoes of both feet;

FIG. 4 is a conceptual diagram for describing a local coordinate system and a world coordinate system in the present disclosure;

FIG. 5 is a conceptual diagram for describing a human body surface set for the human body;

FIG. 6 is a conceptual diagram for describing one gait cycle with the right foot as a reference;

FIG. 7 is a block diagram illustrating an example of a configuration of the determination device in the present disclosure;

FIG. 8 is a conceptual diagram for describing a relative change value calculated by the determination device in the present disclosure;

FIG. 9 illustrates an example of a trajectory in gait along a curve (curve gait) by footprint;

FIG. 10 illustrates an example of a trajectory in gait with staggering (staggering gait) with tracks;

FIG. 11 is a conceptual diagram for describing an example of estimation of a gait situation by the determination device in the present disclosure;

FIG. 12 is a flowchart for describing an example of the operation of the determination device according to the present disclosure;

FIG. 13 is a flowchart for describing an example of a relative change value calculation process by the determination device according to the present disclosure;

FIG. 14 is a block diagram illustrating an example of a configuration of the determination device in the present disclosure;

FIG. 15 is a conceptual diagram illustrating a display example of information according to a gait situation determined by the determination device in the present disclosure;

FIG. 16 is a conceptual diagram illustrating a display example of information according to a gait situation determined by the determination device in the present disclosure;

FIG. 17 is a block diagram illustrating an example of a configuration of the determination device in the present disclosure;

FIG. 18 is a conceptual diagram for describing a relative change value calculated by the determination device in the present disclosure;

FIG. 19 is a conceptual diagram for describing an example of a gait situation to be determined by the determination device in the present disclosure;

FIG. 20 is a conceptual diagram for describing an example of a gait situation to be determined by the determination device in the present disclosure;

FIG. 21 is a conceptual diagram for describing an example of estimation of a gait situation by the determination device in the present disclosure;

FIG. 22 is a flowchart for describing an example of a relative change value calculation process by the determination device according to the present disclosure;

FIG. 23 is a conceptual diagram illustrating a display example of information according to a gait situation determined by the determination device in the present disclosure;

FIG. 24 is a conceptual diagram illustrating a display example of information according to a gait situation determined by the determination device in the present disclosure;

FIG. 25 is a conceptual diagram illustrating a display example of information according to a gait situation determined by the determination device in the present disclosure;

FIG. 26 is a block diagram illustrating an example of a configuration of the determination device in the present disclosure;

FIG. 27 is a flowchart for describing an example of the operation of the determination device according to the present disclosure;

FIG. 28 is a block diagram illustrating an example of a configuration of the determination device in the present disclosure;

FIG. 29 is a conceptual diagram illustrating an example in which a user interface for inputting a tag in the present disclosure is displayed on a screen of a mobile terminal;

FIG. 30 is a flowchart for describing an example of the operation of the determination device according to the present disclosure;

FIG. 31 is a conceptual diagram illustrating a display example of information according to a gait situation determined by the determination device in the present disclosure;

FIG. 32 is a conceptual diagram illustrating a display example of information according to a gait situation determined by the determination device in the present disclosure;

FIG. 33 is a block diagram illustrating an example of a configuration of the determination device in the present disclosure;

FIG. 34 is a flowchart for describing an example of the operation of the determination device according to the present disclosure; and

FIG. 35 is a block diagram illustrating an example of a hardware configuration that executes control and processing in the present disclosure.

### EXAMPLE EMBODIMENT

Example embodiments of the present invention will be described below with reference to the drawings. In the following example embodiments, technically preferable limitations are imposed to carry out the present invention, but the scope of this invention is not limited to the following description. In all drawings used to describe the following example embodiments, the same reference numerals denote similar parts unless otherwise specified. In addition, in the following example embodiments, a repetitive description of similar configurations or arrangements and operations may be omitted. The direction of the arrows in the drawings is for example only and does not limit the direction of data, signals, etc.

### First Example Embodiment

First, an example of a gait measurement system according to a first example embodiment will be described with reference to the drawings. The gait measurement system of the present example embodiment determines the gait situation of the user using the sensor data regarding the motion of the foot according to the gait of the user. The gait situation includes a normal gait and an exceptional gait. The normal gait indicates a linear gait situation on a flat ground. The exceptional gait indicates a gait situation different from the normal gait. For example, the exceptional gait includes a gait on a meandering road, a stair, or a slope. In the present example embodiment, an example of determines a non-linear exceptional gait and a normal gait on a flat ground will be described.

### Configuration

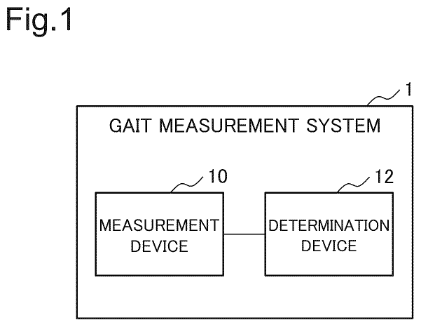

FIG. 1 is a block diagram illustrating an example of a configuration of a gait measurement system in the present disclosure; A gait measurement system 1 includes a measurement device 10 and a determination device 12. For example, the measurement device 10 is installed at footwear of a subject (user) whose physical condition is to be estimated. For example, the function of the determination device 12 is installed in a mobile terminal carried by a subject (user). Hereinafter, configurations of the measurement device 10 and the determination device 12 will be individually described.

### Measurement Device

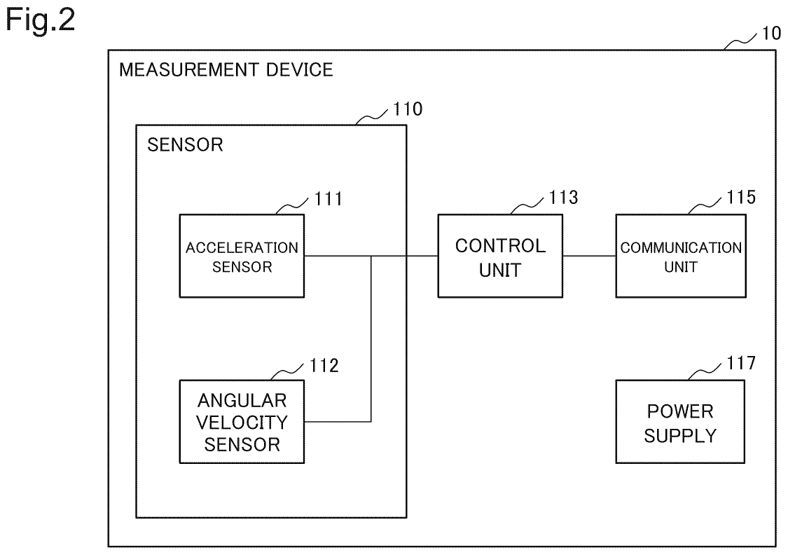

FIG. 2 is a block diagram illustrating an example of a configuration of a measurement device in the present disclosure; The measurement device 10 includes a sensor 110, a control unit 113, a communication unit 115, and a power supply 117. The sensor 110 includes an acceleration sensor 111 and an angular velocity sensor 112. The sensor 110 may include a sensor other than the acceleration sensor 111 and the angular velocity sensor 112. The sensor, other than the acceleration sensor 111 and the angular velocity sensor 112, that can be included in the sensor 110 will not be described.

The acceleration sensor 111 is a sensor that measures acceleration (also referred to as spatial acceleration) in the three axial directions. The acceleration sensor 111 measures acceleration as a physical quantity related to the motion of the foot. The acceleration sensor 111 outputs the measured acceleration to the control unit 113. For example, a sensor of a piezoelectric type, a piezoresistive type, a capacitance type, or the like can be used as the acceleration sensor 111. The sensor used as the acceleration sensor 111 is not limited as long as it can measure acceleration.

The angular velocity sensor 112 is a sensor that measures angular velocities around the three axes (also referred to as spatial angular velocities). The angular velocity sensor 112 measures an angular velocity as a physical quantity related to the motion of the foot. Angular velocity sensor 112 outputs the measured angular velocity to control unit 113. For example, a sensor of a vibration type, a capacitance type, or the like can be used as the angular velocity sensor 112. The sensor used as the angular velocity sensor 112 is not limited as long as the sensor can measure the angular velocity.

The sensor 110 is achieved by, for example, an inertial measurement device that measures acceleration and angular velocity. An example of the inertial measurement device is an inertial measurement unit (IMU). The IMU includes the acceleration sensor 111 that measures acceleration in three axial directions and the angular velocity sensor 112 that measures angular velocities around the three axes. The sensor 110 may be achieved by an inertial measurement device such as a vertical gyro (VG) or an attitude heading reference system (AHRS). The sensor 110 may be achieved by a global positioning system/inertial navigation system (GPS/INS). The sensor 110 may be achieved by a device other than the inertial measurement device as long as it can measure a physical quantity related to the motion of the foot.

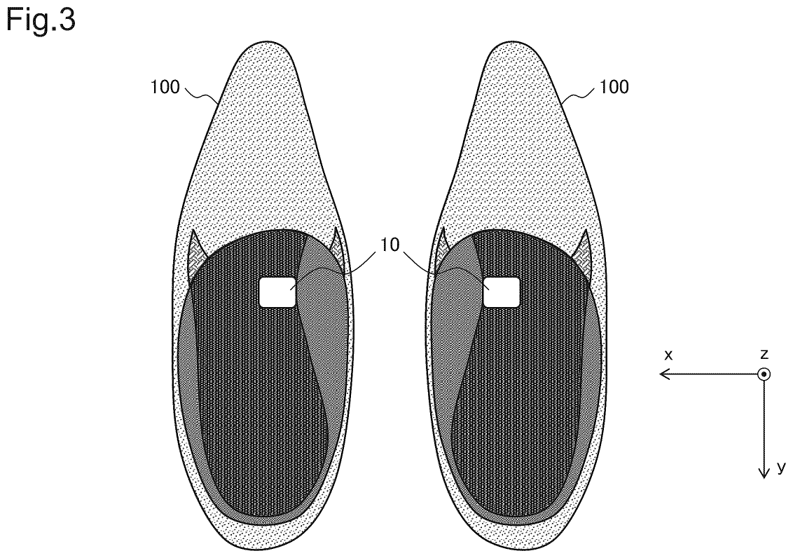

FIG. 3 is a conceptual diagram illustrating an example in which the measurement device in the present disclosure is disposed in shoes of both feet; In the example of FIG. 3, the measurement device 10 is installed at a position related to the back side of the arch of foot. For example, the measurement device 10 is disposed in an insole inserted into the shoe 100. For example, the measurement device 10 may be disposed on the bottom face of a shoe 100. For example, the measurement device 10 may be embedded in the main body of the shoe 100. The measurement device 10 may be detachable from the shoe 100 or may not be detachable from the shoe 100. The measurement device 10 may be installed at a position other than the back side of the arch of foot as long as the sensor data related to the motion of the foot can be measured. The measurement device 10 may be installed on a sock worn by the user or a decorative article such as an anklet worn by the user. The measurement device 10 may be directly attached to the foot or may be embedded in the foot. As long as the data from which the physical condition can be estimated can be measured, the measurement device 10 may be disposed in one shoe 100.

In the example of FIG. 3, a local coordinate system including an x axis in the left-right direction, a y axis in the front-rear direction, and a z axis in the vertical direction is set with the measurement device 10 (sensor 110) as a reference. FIG. 3 illustrates an example in which the same coordinate system is set for the left foot and the right foot. For example, in a case where the sensors 110 produced with the same specifications are disposed in the left and right shoes 100, the vertical directions (directions in the Z axis direction) of the sensors 110 disposed in the left and right shoes 100 are the same. In this case, the three axes of the local coordinate system set in the sensor data derived from the left foot and the three axes of the local coordinate system set in the sensor data derived from the right foot are the same for the left and right feet. In the present disclosure, the left side of the x axis is positive, the front side of the y axis is positive, and the upper side of the z axis is positive. The positive and negative directions of the x axis, the y axis, and the z axis are set in any direction.

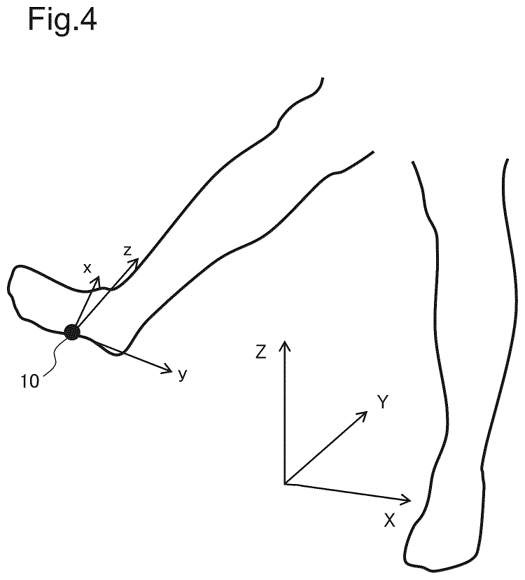

FIG. 4 is a conceptual diagram for describing a local coordinate system and a world coordinate system in the present disclosure; The world coordinate system (X axis, Y axis, Z axis) is set with respect to the ground. The local coordinate system (x axis, y axis, z axis) is set for the measurement device. In the world coordinate system (X axis, Y axis, Z axis), the X axis is set in a lateral direction of a user, the Y axis is set in a front-rear direction of the user, and the Z axis is set in a vertical direction in a state in which the user facing the traveling direction is upright. The example of FIG. 4 conceptually illustrates the relationship between the local coordinate system (x axis, y axis, z axis) and the world coordinate system (X axis, Y axis, Z axis), and does not accurately illustrate the relationship between the local coordinate system and the world coordinate system that varies depending on a gait of the user.

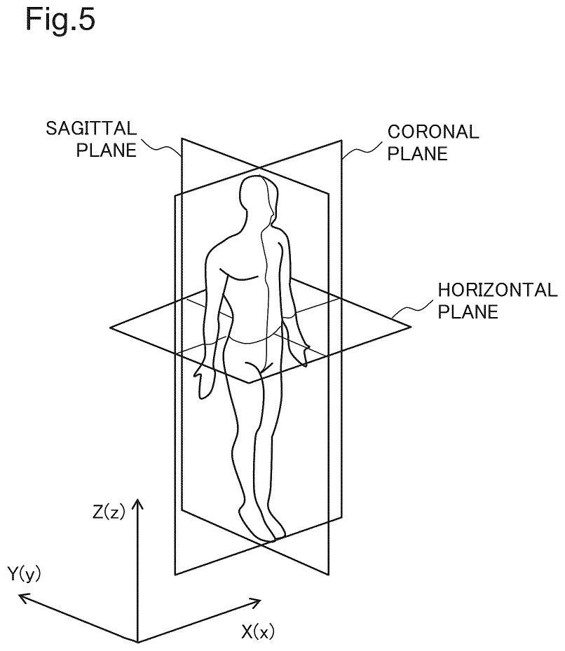

FIG. 5 is a conceptual diagram for describing a human body surface set for the human body; In the present example embodiment, a sagittal plane, a coronal plane, and a horizontal plane are defined. The sagittal plane is a human body surface that divides the body into right and left. The coronal plane is a human body surface that divides the body back and forth. The horizontal plane is a human body surface that horizontally divides the body. As illustrated in FIG. 5, the world coordinate system and the local coordinate system coincide with each other in a state in which the user is standing upright with the center line of the foot being directed in the traveling direction. FIG. 5 illustrates an example in which different coordinate systems are set for the left foot and the right foot. In the present example embodiment, rotation in the sagittal plane with the X axis (x axis) as the rotation axis is defined as roll, rotation in the coronal plane with the Y axis (y axis) as the rotation axis is defined as pitch, and rotation in the horizontal plane with the Z axis (z axis) as the rotation axis is defined as yaw. A rotation angle in a sagittal plane with the X axis (x axis) as a rotation axis is defined as a roll angle, a rotation angle in a coronal plane with the Y axis (y axis) as a rotation axis is defined as a pitch angle, and a rotation angle on a horizontal plane with the Z axis (z axis) as a rotation axis is defined as a yaw angle.

The control unit 113 causes the acceleration sensor 111 and the angular velocity sensor 112 to measure sensor data. For example, the control unit 113 causes the acceleration sensor 111 and the angular velocity sensor 112 to start measurement in response to a measurement start signal transmitted from the determination device 12. For example, the control unit 113 may cause the acceleration sensor 111 and the angular velocity sensor 112 to start measurement at a timing when the gait by the user is detected. For example, after the heights of both feet in the vertical direction are the same over a predetermined period set in advance, the control unit 113 starts the measurement of the sensor data from the time point at which the motion of one of the right and left feet in the traveling direction is detected as a starting point. The control unit 113 may be configured to start measurement of sensor data at a predetermined timing set in advance.

The control unit 113 acquires accelerations in three axial directions from the acceleration sensor 111. The control unit 113 acquires angular velocities around the three axes from angular velocity sensor 112. For example, the control unit 113 performs analog-to-digital conversion (AD conversion) on the acquired physical quantities (analog data) such as angular velocity and acceleration. The physical quantity (analog data) measured by each of the acceleration sensor 111 and the angular velocity sensor 112 may be converted into digital data in each of the acceleration sensor 111 and the angular velocity sensor 112. For example, an AD conversion circuit that performs AD conversion on physical quantities (analog data) such as the angular velocity and the acceleration may be provided. The control unit 113 outputs the converted digital data (also referred to as sensor data) to the communication unit 115. For example, the control unit 113 may temporarily store the sensor data in a storage unit (not illustrated).

The sensor data includes at least acceleration data converted into digital data and angular velocity data converted into digital data. The acceleration data includes acceleration vectors in the three axial directions. The angular velocity data includes angular velocity vectors around the three axes. The acceleration data and the angular velocity data are associated with acquisition time of the data. The control unit 113 may add correction such as a mounting error, temperature correction, and linearity correction to the acceleration data and the angular velocity data.

For example, the control unit 113 is achieved by a microcomputer or a microcontroller that performs overall control and data processing of the measurement device 10. For example, the control unit 113 includes a central processing unit (CPU), a random access memory (RAM), a read only memory (ROM), a flash memory, and the like.

The communication unit 115 acquires sensor data from the control unit 113. The communication unit 115 transmits the acquired sensor data to the determination device 12. The sensor data transmitted from the communication unit 115 is received by the determination device 12. The transmission timing of the sensor data is not particularly limited. For example, the communication unit 115 transmits sensor data at a transmission timing set in advance. For example, the communication unit 115 transmits the sensor data in real time in response to the measurement of the sensor data. For example, the communication unit 115 may store sensor data measured during a predetermined period and collectively transmit the stored sensor data at a timing set in advance. For example, the communication unit 115 may be configured to receive a measurement start signal from the determination device 12. In this case, the communication unit 115 outputs the received measurement start signal to the control unit 113.

For example, the communication unit 115 transmits the sensor data to the determination device 12 via wireless communication. For example, the communication unit 115 transmits sensor data to the determination device 12 via a wireless communication function (not illustrated) conforming to a standard such as Bluetooth (registered trademark) or WiFi (registered trademark). The communication function of the communication unit 115 may conform to a standard other than Bluetooth (registered trademark) or WiFi (registered trademark). The communication unit 115 may transmit the sensor data to the determination device 12 via a wire such as a cable.

The power supply 117 is a battery that supplies power for the measurement device 10 to operate. For example, the power supply 117 is achieved by a thin battery such as a coin type or a button type. For example, the power supply 117 is achieved by a primary battery such as a lithium primary battery, a silver oxide battery, an alkaline button battery, or an air zinc battery. In the case of being achieved by the primary battery, the power supply 117 is preferably achieved by a long-life battery. The power supply 117 may be achieved by a rechargeable secondary battery. In the case of being achieved by the secondary battery, the power supply 117 may be a battery that can be charged in a wired manner or may be a battery that can wirelessly supply power. When the power supply 117 can wirelessly supply power, the wireless power supply device may be disposed at a place where footwear is placed, such as an entrance or a footwear box. When the footwear on which the measurement device 10 is mounted is disposed on the wireless power supply device, the measurement device 10 can be charged appropriately when not in use.

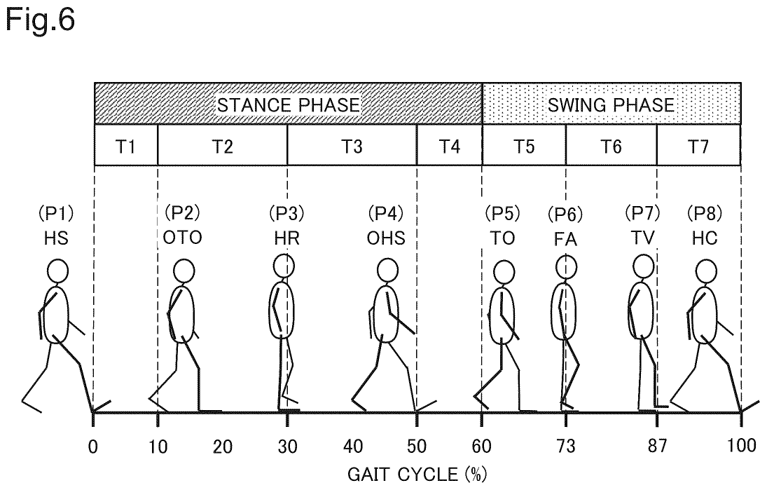

FIG. 6 is a conceptual diagram for describing one gait cycle with the right foot as a reference; One gait cycle based on the left foot is also similar to that of the right foot. The horizontal axis of FIG. 6 illustrates one gait cycle of the right foot with a time point at which the heel of the right foot lands on the ground as a starting point and a time point at which the heel of the right foot next lands on the ground as a terminal point. The horizontal axis in FIG. 6 is normalized with one gait cycle as 100%. Normalizing one gait cycle by 100% is referred to as first normalization. The one gait cycle of one foot is roughly divided into a stance phase in which at least part of the back side of the foot is in contact with the ground and the swing phase in which the back side of the foot is away from the ground. The stance phase is a period in which at least part of the back side of the foot is in contact with the ground. The stance phase is further subdivided into an initial stance period T1, a mid-stance period T2 of standing, a terminal stance period T3 of standing, and a pre-swing period T4. The swing phase is a period in which the back side of the foot is away from the ground. The swing phase is further subdivided into an initial swing period T5, a mid-swing period T6, and a terminal swing period T7. The horizontal axis of FIG. 6 is normalized in such a way that the stance phase is 60% and the swing phase is 40%. Normalizing the gait waveform in such a way that the stance phase is 60% and the swing phase is 40% is referred to as second normalization. The period illustrated in FIG. 6 is an example, and does not limit the periods constituting one gait cycle, the names of these periods, and the like.

As illustrated in FIG. 6, a plurality of events occurs during the gait. In gait, a plurality of events in gait is also referred to as gait events. P1 represents an event (heel strike (HS)) in which the heel of the right foot is grounded. P2 represents an event (opposite toe off (OTO)) in which the toe of the right foot is away from the ground while the sole of the left foot is grounded. P3 represents an event (heel rise (HR)) in which the heel of the right foot is lifted while the sole of the right foot is grounded. P4 represents an event (opposite heel strike (OHS)) in which the heel of the left foot is grounded. P5represents an event (toe off (TO)) in which the toe of the left foot is away from the ground while the sole of the right foot is grounded. P6 represents an event (foot adjacent (FA)) in which the left foot and the right foot cross each other while the sole of the left foot is grounded. P7 represents an event (tibia vertical (TV)) in which the tibia of the left foot is substantially perpendicular to the ground in a state where the sole of the right foot is in contact with the ground. P8 represents an event (heel strike (HS)) in which the heel of the right foot is grounded. P8 corresponds to the terminal point of the gait cycle starting from P1 and corresponds to the starting point of the next gait cycle. The gait event illustrated in FIG. 6 is an example, and does not limit events that occur during gait or names of these events.

The timing of the heel strike is the timing of the minimum peak immediately after the maximum peak appearing in the time-series data of the traveling direction acceleration (Y direction acceleration). The maximum peak serving as a mark of the timing of the heel strike corresponds to the maximum peak of the gait waveform for one gait cycle. A section between the successive heel strikes corresponds to one gait cycle. The timing of the toe off is the rising timing of the maximum peak appearing after the period of the stance phase in which the fluctuation does not appear in the time-series data of the traveling direction acceleration (Y direction acceleration). The timing at the midpoint between the timing at which the roll angle is minimum and the timing at which the roll angle is maximum corresponds to the mid-stance period.

### Determination Device

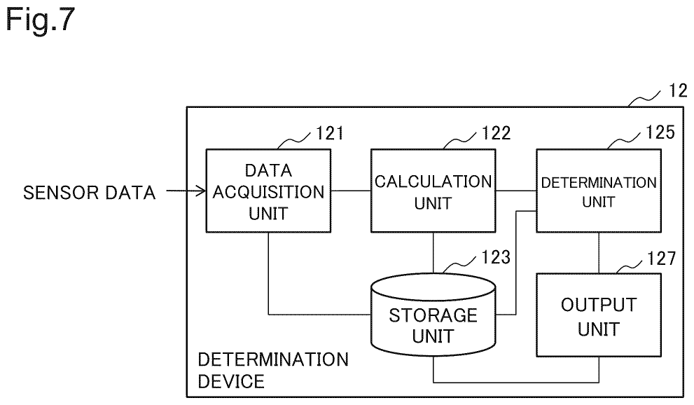

FIG. 7 is a block diagram illustrating an example of a configuration of the determination device in the present disclosure; The determination device 12 includes a data acquisition unit 121, a calculation unit 122, a storage unit 123, a determination unit 125, and an output unit 127.

The data acquisition unit 121 acquires time-series data of sensor data from the measurement device 10. The data acquisition unit 121 receives the time-series data of the sensor data from the measurement device 10 via wireless communication. For example, the data acquisition unit 121 receives the time-series data of the sensor data from the measurement device 10 via a wireless communication function (not illustrated) conforming to a standard such as Bluetooth (registered trademark) or WiFi (registered trademark). The communication function of the data acquisition unit 121 may conform to a standard other than Bluetooth (registered trademark) or WiFi (registered trademark) as long as communication with the measurement device 10 can be performed. The data acquisition unit 121 may receive the time-series data of the sensor data from the measurement device 10 via a wire such as a cable.

The calculation unit 122 extracts an end point of the gait cycle from the time-series data of the sensor data. A section between the successive end points corresponds to one gait cycle. Among two successive end points, an end point preceding in time series is set as a start point of one gait cycle. Among two successive end points, a later end point in time series is set as a terminal point of one gait cycle. For example, the end point of the gait cycle is set to the timing of the mid-stance period or heel strike. The timing at the midpoint between the timing at which the roll angle is minimum and the timing at which the roll angle is maximum corresponds to the mid-stance period. In this case, a section between the successive mid-stance periods corresponds to one gait cycle. The timing of the heel strike is the timing of the minimum peak immediately after the maximum peak appearing in the time-series data of the traveling direction acceleration (Y direction acceleration). The maximum peak serving as a mark of the timing of the heel strike corresponds to the maximum peak of the gait waveform for one gait cycle. Each of a section between the successive mid-stance periods and a section between the successive heel strikes corresponds to one gait cycle. In this case, a section between the successive heel strikes corresponds to one gait cycle. The timing of the mid-stance period or the heel strike is an example, and does not limit the end point of the gait cycle. For example, the end point of the gait cycle may be set to the timing of a gait event such as a toe off, an opposite toe off, a heel rise, an opposite heel strike, a toe off, a foot adjacent, and a tibia vertical. A method of detecting the timing of the gait event will not be described.

The calculation unit 122 extracts time-series data of sensor data between two successive end points as a gait waveform for one gait cycle. The calculation unit 122 may normalize the extracted gait waveform. For example, the calculation unit 122 normalizes (first normalizes) the time of the extracted gait waveform for one gait cycle to a gait cycle of 0 to 100% (percent). A section such as 1% or 10% included in the 0 to 100% gait cycle is also referred to as a gait phase. For example, the calculation unit 122 normalizes (second normalizes) the first normalized gait waveform for one gait cycle in such a way that the stance phase is 60% and the swing phase is 40%. When the gait waveform is second normalized, it is possible to reduce the shift of the gait phase from which the feature amount used for estimation of the gait state is extracted.

For example, the calculation unit 122 extracts a gait waveform for one gait cycle using the traveling direction acceleration (Y direction acceleration). In this case, the calculation unit 122 extracts a gait waveform for one gait cycle in accordance with the gait cycle of the traveling direction acceleration (Y direction acceleration) with respect to acceleration/angular velocity/angle other than the traveling direction acceleration (Y direction acceleration). The calculation unit 122 extracts a gait waveform related to the acceleration in three axial directions, a gait waveform related to the angular velocity around the three axes, and a gait waveform related to the angle around the three axes. The calculation unit 122 may generate time-series data of angles around the three axes by integrating time-series data of angular velocities around the three axes. The calculation unit 122 normalizes the extracted gait waveform for one gait cycle.

The calculation unit 122 may extract a gait waveform for one gait cycle using acceleration/angular velocity other than the traveling direction acceleration (Y direction acceleration). For example, the calculation unit 122 detects the heel strike and the toe off from the time-series data of the vertical direction acceleration (Z direction acceleration). The timing of the heel strike is a timing of a steep minimum peak appearing in the time-series data of the vertical direction acceleration (Z direction acceleration). At the timing of the steep minimum peak, the value of the vertical direction acceleration (Z direction acceleration) is substantially zero. The minimum peak serving as a mark of the timing of the heel strike corresponds to the minimum peak of the gait waveform data for one gait cycle. A section between the successive heel strikes is one gait cycle. The timing of the toe off is a timing of an inflection point in the middle of gradually increasing after the time-series data of the vertical direction acceleration (Z direction acceleration) passes through a section with a small fluctuation after the maximum peak immediately after the heel strike. The calculation unit 122 may extract a gait waveform for one gait cycle using both the traveling direction acceleration (Y direction acceleration) and the vertical direction acceleration (Z direction acceleration). The calculation unit 122 may extract the gait waveform for one gait cycle using acceleration, angular velocity, angle, and the like other than the traveling direction acceleration (Y direction acceleration) and the vertical direction acceleration (Z direction acceleration).

The calculation unit 122 calculates a relative change value indicating a relative change from the traveling axis on the horizontal plane for each gait cycle using the time-series data (gait waveform) of the sensor data. The calculation unit 122 calculates a relative change value from a start point to a terminal point on the horizontal plane (XY plane) using the extracted gait waveform. The relative change value on the horizontal plane (XY plane) indicates a relative change from the traveling axis at the start point.

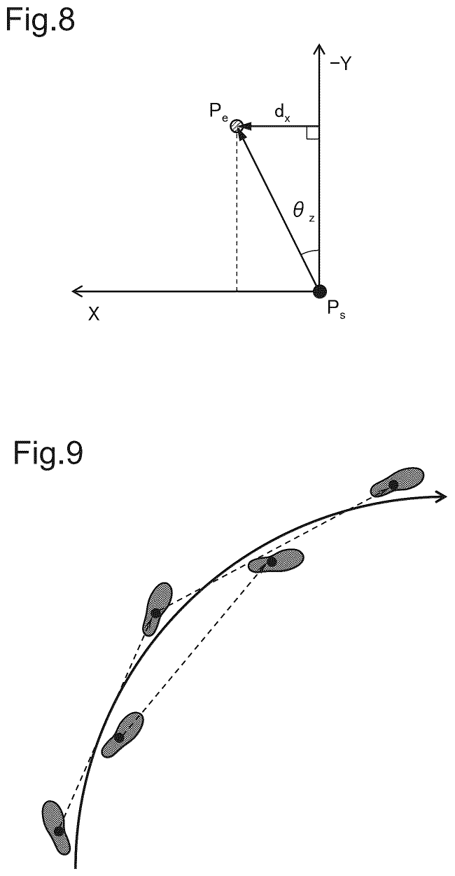

FIG. 8 is a conceptual diagram for describing a relative change value calculated by the determination device in the present disclosure; FIG. 8 illustrates a relative change value of one gait cycle (one stride) from the start point Pₛ to the terminal point Pₑ. The relative change value includes a relative displacement dₓ and a relative angle θ_{z}. The relative angle θ_{z} corresponds to an angle formed by the traveling axis (−Y) at the start point Pₛ and a straight line passing through the start point and the terminal point on the horizontal plane (XY plane). The relative displacement dₓ corresponds to a distance in a horizontal plane (XY plane) between the traveling axis (−Y) at the start point Pₛ and the terminal point Pₑ. The calculation unit 122 calculates a relative angle θ_{z} formed by the traveling axis at the start point and a straight line passing through the start point and the terminal point on the horizontal plane. The calculation unit 122 calculates the relative displacement dₓ in the left-right direction from the start point to the terminal point. The calculation unit 122 stores the calculated relative angle θ_{z} and relative displacement dₓ in the storage unit 123.

The storage unit 123 stores the sensor data acquired by the data acquisition unit 121. The storage unit 123 stores the relative angle θ_{z} and the relative displacement dₓ calculated by the calculation unit 122. As will be described later, the storage unit 123 stores the gait situation determined by the determination unit 125.

The determination unit 125 acquires the relative change value in the target period from the storage unit 123. The target period is a time zone over a plurality of gait cycles (stride). The determination unit 125 determines the gait situation in the target period using the time-series data of the relative change value. For example, the determination unit 125 determines the gait situation in the target period using the three-dimensional rotation correction performed using the relative change value. For example, the determination unit 125 determines the gait situation in the target period using a machine learning model (determination model) generated by machine learning. The gait situation includes a straight gait (normal gait), a gait along a curve (curve gait), and a gait with staggering (staggering gait). The gait situation is not limited to the example described herein as far as gait on a horizontal plane is concerned. The determination unit 125 records the gait situation in the target period in association with the sensor data measured in the gait cycle included in the target period.

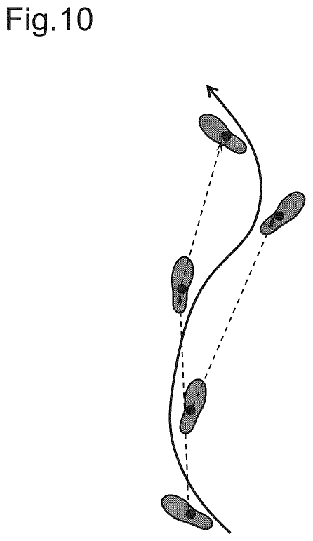

FIGS. 9 to 10 are conceptual diagrams for describing an example of a gait situation to be determined by the determination device in the present disclosure. FIGS. 9 to 10 are diagrams when viewed from above. FIG. 9 illustrates an example of a trajectory in gait along a curve (curve gait) by footprint; FIG. 10 illustrates an example of a trajectory in gait with staggering (staggering gait) with tracks; FIGS. 9 to 10 are examples, and do not limit the gait situation to be determined by the determination device 12. For example, a gait situation such as crawling, claudication, and cane gait may be included in the determination target by the determination device 12.

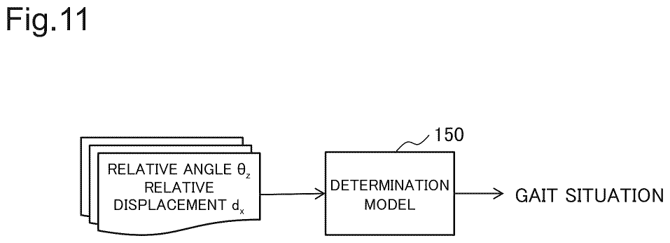

FIG. 11 is a conceptual diagram for describing an example of estimation of a gait situation by the determination device in the present disclosure; A determination model 150 is a machine learning model generated by machine learning. For example, the determination model 150 is a model obtained by learning a data set having the relative angle θ_{z} and the relative displacement dₓ as explanatory variables and the gait situation as an objective variable as teacher data. The determination model 150 outputs the gait situation according to the input of the time-series data of the relative angle θ_{z} and the relative displacement dₓ calculated using the sensor data measured in the target period. The determination model 150 may be stored in an external storage device constructed in a cloud, a server, or the like. In this case, the determination unit 125 uses the determination model 150 via an interface (not illustrated) connected to the storage device.

For example, the determination model 150 is a learning model trained using a convolutional neural network (CNN) method. For example, the determination model 150 is a model trained using a principal component analysis (PCA) method. For example, the determination model 150 is a learning model trained using a variational autoencoder (VAE). For example, the determination model 150 is a learning model trained using a conditional generative adversarial networks (GAN) method. For example, the determination model 150 may be generated by training using a linear regression algorithm. For example, the determination model 150 may be generated by training using an algorithm of a support vector machine (SVM). For example, the determination model 150 may be generated by training using a Gaussian process regression (GPR) algorithm. For example, the determination model 150 may be generated by training using a random forest (RF) algorithm. The above method is an example, and does not limit the method of training the determination model 150.

The output unit 127 outputs the sensor data stored in the storage unit 123. The sensor data is associated with the gait situation at the measured time point. Information including sensor data associated with a gait situation is also referred to as gait information. For example, the output unit 127 outputs the gait information to a mobile terminal carried by the subject. For example, the output unit 127 outputs the gait information to a terminal device or a server using the sensor data via a mobile terminal carried by the subject. For example, the output unit 127 may be configured to output sensor data to an external system or the like that uses the sensor data.

For example, the determination device 12 is constructed in a cloud or a server connected to a mobile terminal carried by the subject via a communication network. The mobile terminal is a portable communication device. For example, the mobile terminal is a portable communication device having a communication function, such as a smartphone, a smart watch, or a mobile telephone. For example, the determination device 12 is connected to a mobile terminal via wireless communication. For example, the determination device 12 is connected to a mobile terminal via a wireless communication device (not illustrated) conforming to a standard such as Bluetooth (registered trademark) or WiFi (registered trademark). The wireless communication device may conform to a standard other than Bluetooth (registered trademark) or WiFi (registered trademark). The corrected gait data may be used by an application installed at the mobile terminal. For example, the mobile terminal executes processing using the sensor data by an application installed in the mobile terminal.

The determination device 12 may be configured to calculate a gait index. For example, the determination device 12 calculates the gait index using the normalized gait waveform. The gait index is used for estimation of a physical condition, physical ability, and the like. The gait index calculated by the determination device 12 is not particularly limited. For example, the determination device 12 calculates a gait index regarding a distance, a height, an angle, a speed, a time, a center of pressure exclusion index (CPEI), a frail level, and the like.

For example, the determination device 12 calculates an index related to a distance and a height as a gait index. For example, the determination device 12 calculates a stride length, an outward turning distance, a foot raising height, a foot clearance (FTC), and a minimum toe clearance (MTC). The stride length indicates a distance between a front foot and a rear foot during gait. The outward turning distance indicates the maximum value of the distance at which the foot is away outward with respect to the traveling direction in the swing phase. The foot raising height indicates the maximum value of the distance between the measurement device 10 (sensor 110) and the ground in the swing phase. FTC indicates the maximum value of the distance between the heel and the ground in the swing phase. The MTC indicates the minimum value of the distance between the toe and the ground in the swing phase.

For example, the determination device 12 calculates an index related to an angle as a gait index. For example, the determination device 12 calculates a grounding angle, a ground off angle, a toe direction, a heel strike roll angle, a toe off roll angle, a swing peak angular velocity, and a hallux angle. The grounding angle indicates a maximum value of an angle formed by the back face of the foot and the ground at the time of heel strike. The ground off angle indicates an angle formed by the back face of the foot and the ground in the swing phase. The direction of the toe indicates an average value of the directions of the toe with respect to the traveling direction in the swing phase. The roll angle of the heel strike is an angle formed by the ankle and the ground at the time of the heel strike when viewed from the rear. The roll angle of the toe off ground is an angle formed by the ankle and the ground at the time of kicking when viewed from the rear. The swing peak angular velocity is an angular velocity in the ankle joint dorsiflexion direction in a section from immediately after kicking until the toe comes into closest contact with the ground. The hallux angle indicates an angle at which the thumb of the foot is inclined toward the index finger. Specifically, the hallux angle is an angle formed by the center line of the first metatarsal and the center line of the first proximal phalange.

For example, the determination device 12 calculates an index related to the speed as the gait index. For example, the determination device 12 calculates a gait speed, cadence, and the maximum speed in swing. The gait speed indicates a speed in gait. The cadence indicates the number of steps per minute. The maximum speed in swing indicates a speed at which the user swings out the leg in the swing phase.

For example, the determination device 12 calculates an index related to time as the gait index. For example, the determination device 12 calculates a stance time, a load time, a plantar grounding time, a kicking time, a swing time, and a double support time (DST). The stance time indicates a time during which the foot is in contact with the ground during gait. The stance time is a sum of a load time, a plantar grounding time, and a kicking time. The load time is a time from when the heel is in contact with the ground to when the toe is in contact with the ground in the stance phase. The plantar grounding time is a time during which the entire plantar surface is in contact with the ground and the plantar surface and the ground are horizontal in the stance phase. The kicking time is a time until the toe kicks the ground from the state of the plantar grounding in the stance phase. The swing time indicates a time during which the foot is away from the ground during gait. The DST is divided into a DST1 and a DST2. The DST1 indicates a time during which the foot on which the measurement device 10 (sensor 110) is mounted is in front of the opposite foot in a period in which both feet are simultaneously grounded to the ground. The DST2 indicates a time during which the foot on which the measurement device 10 (sensor 110) is mounted is behind the opposite foot in a period in which both feet are simultaneously grounded to the ground.

For example, the determination device 12 calculates a center of pressure exclusion index (CPEI) as a gait index. The CPEI indicates an estimated value of the expansion ratio of the movement of the foot pressure center portion applied to the ground during the stance phase.

For example, the determination device 12 calculates a frail level as the gait index. The frail level is an estimated value of the frail state according to the gait state. For example, the determination device 12 estimates an index indicating a determination result regarding frailing as the frail level. In a case where there is no possibility of frailing, the determination device 12 estimates an index indicating no frailing. In a case where there is a possibility of frailing, the determination device 12 estimates an index indicating that there is a possibility of frailing. In a case where the possibility of frailing is high, the determination device 12 estimates an index indicating that the possibility of frailing is high.

The determination device 12 may extract a feature amount used for calculation and estimation of the gait index from the gait waveform. For example, the determination device 12 extracts a feature amount for each gait phase cluster according to a condition set in advance. The gait phase cluster is a cluster in which temporally continuous gait phases are integrated. The gait phase cluster includes at least one gait phase. The gait phase cluster also includes a single gait phase. The determination device 12 may extract the physical ability feature amount used for estimating the physical ability. For example, the physical ability feature amount is used to estimate at least one of physical ability such as grip strength (total muscle strength of the whole body), dynamic balance, lower limb muscle strength, mobility, and static balance.

### Operation

Next, an operation of the gait measurement system 1 will be described with reference to the drawings. Hereinafter, the operation of the determination device 12 included in the gait measurement system 1 will be described.

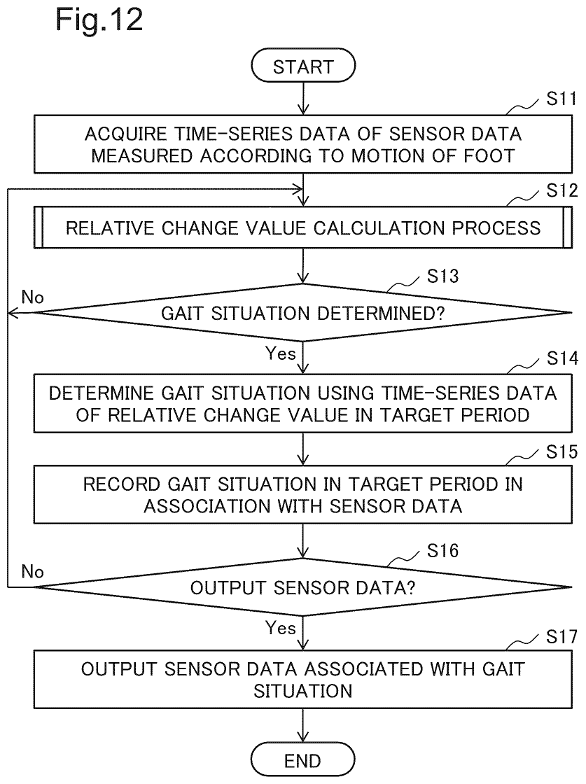

FIG. 12 is a flowchart for describing an example of the operation of the determination device according to the present disclosure; In the description of the processing along the flowchart of FIG. 12, the components of the determination device 12 will be described as the operation subject. The operation subject of the processing along the flowchart of FIG. 12 may be the determination device 12.

In FIG. 12, first, the data acquisition unit 121 acquires time-series data of sensor data measured according to the motion of the foot (step S11).

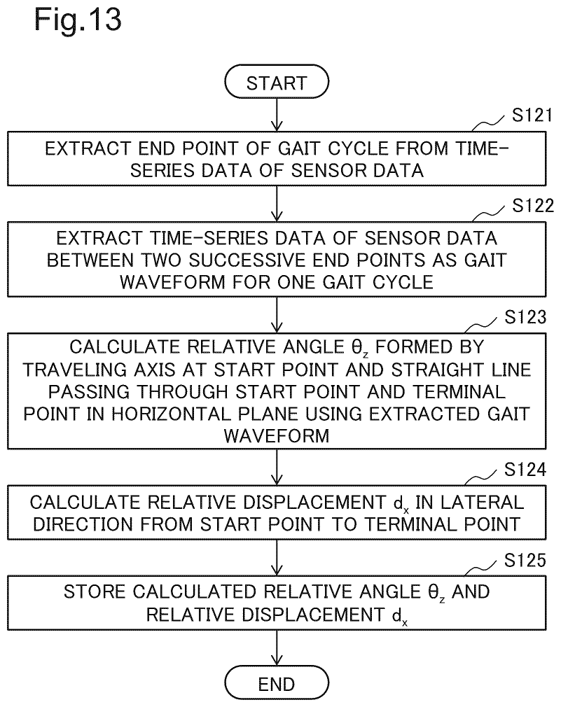

Next, the calculation unit 122 executes a relative change value calculation process (step S12). Details of the relative change value calculation process will be described later (FIG. 13).

In a case where the gait situation is determined (Yes in step S13), the determination unit 125 determines the gait situation using the time-series data of the relative change value in the target period (step S14). On the other hand, in a case where the gait situation is not determined (No in step S13), the process returns to step S12.

After step S14, the storage unit 123 records the gait situation in the target period in association with the sensor data (step S15).

In a case where the sensor data is output (Yes in step S16), the output unit 127 outputs the sensor data associated with the gait situation (step S17). On the other hand, in a case where the sensor data is not output (No in step S16), the process returns to step S12.

### Relative Change Value Calculation Process

Next, the relative change value calculation process (step S12 in FIG. 12) by the determination device 12 will be described in detail with reference to the drawings.

FIG. 13 is a flowchart for describing an example of a relative change value calculation process by the determination device according to the present disclosure; In the description of the processing along the flowchart of FIG. 13, the components of the determination device 12 will be described as the operation subject. The operation subject of the process along the flowchart of FIG. 13 may be the determination device 12.

In FIG. 13, first, the calculation unit 122 extracts an end point of the gait cycle from the time-series data of the sensor data (step S121).

Next, the calculation unit 122 extracts time-series data of sensor data between two successive end points as a gait waveform for one gait cycle (step S122).

Next, the calculation unit 122 calculates a relative angle θ_{z} formed by the traveling axis at the start point and the straight line passing through the start point and the terminal point on the horizontal plane using the extracted gait waveform (step S123).

Next, the calculation unit 122 calculates a relative displacement dₓ in the lateral direction from the start point to the terminal point (step S124). The order of the processing in step S123 and the processing in step S124 may be switched.

Next, the storage unit 123 stores the calculated relative angle θ_{z} and relative displacement dₓ (step S125). The relative angle θ_{z} and the relative displacement dx stored in the storage unit 123 are used for determining the gait situation.

### Application Example

Next, an application example according to the present example embodiment will be described with reference to the drawings. In the present application example, information according to the gait situation is generated using the sensor data processed by the determination device 12. The generated information is output to a mobile terminal carried by the user.

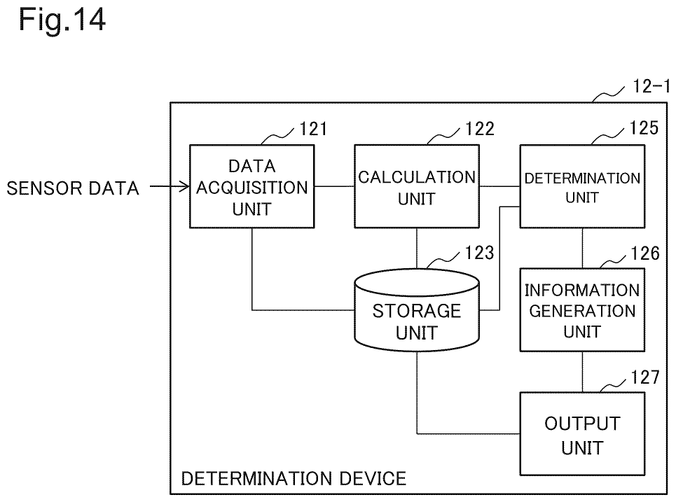

FIG. 14 is a block diagram illustrating an example of a configuration of the determination device in the present disclosure; A determination device 12-1 includes the data acquisition unit 121, the calculation unit 122, the storage unit 123, the determination unit 125, an information generation unit 126, and the output unit 127. The determination device 12-1 is similar to the determination device 12 except that it includes the information generation unit 126. Components other than the information generation unit 126 will not be described.

The information generation unit 126 acquires sensor data associated with the gait situation. The information generation unit 126 may be configured to acquire a gait index generated using the sensor data. The information generation unit 126 generates information according to the gait situation using the sensor data associated with the gait situation. For example, the information processing unit generates information according to the gait situation of the user using a trained machine learning model constructed in advance. For example, the information processing unit may be configured to generate information according to the gait situation of the user using large language models (LLM). For example, the information generation unit 126 generates information according to the gait situation. For example, the information generation unit 126 generates action recommendation information for recommending an action according to the gait situation. The information generated by the information generation unit 126 is output by the output unit 127. As will be described later, the information output by the output unit 127 is displayed on a screen of a mobile terminal carried by the user, or is output by voice by the mobile terminal.

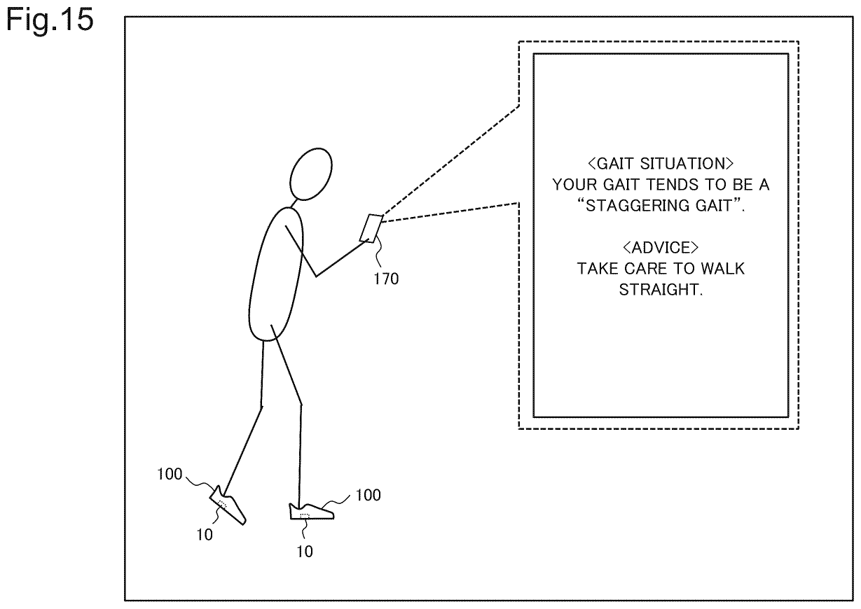

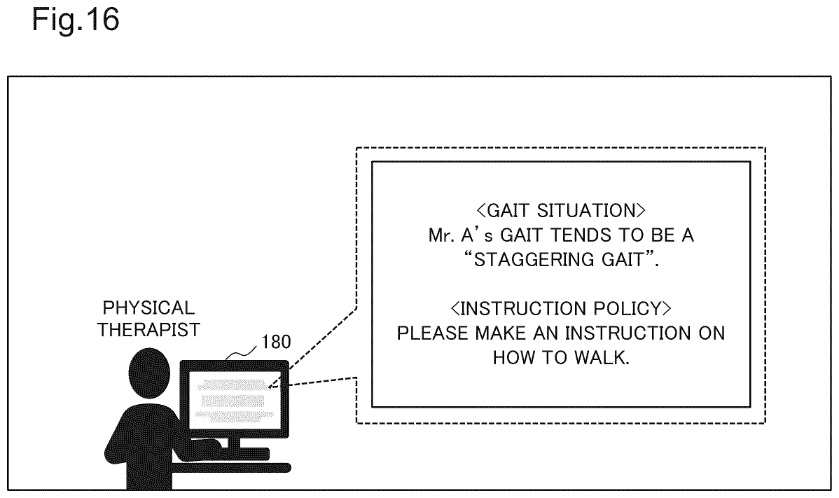

FIGS. 15 to 16 are conceptual diagrams illustrating display examples of information according to the gait situation determined by the determination device in the present disclosure. FIG. 15 illustrates an example in which information related to the gait situation of the user is displayed on the screen of a mobile terminal 170 carried by the user who wears the shoes 100 on which the measurement device 10 is disposed and walks. FIG. 16 illustrates an example in which information related to the gait situation of the user is displayed on the screen of a terminal device 180 used by the physical therapist who is in charge of the physical condition of the user.

In the example of FIG. 15, information that “Your gait tends to be a “staggering gait”” is displayed on the screen of the mobile terminal 170 according to the gait situation determined regarding the user. In the example of FIG. 15, action recommendation information that “Take care to walk straight” is displayed on the screen of the mobile terminal 170 according to the gait situation determined regarding the user. The action recommendation information is optimized in accordance with the gait situation of the user. The action recommendation information includes information for urging the user to make a decision. The user who has confirmed the information displayed on the screen of the mobile terminal 170 can try to improve the gait situation by taking action according to the action recommendation information.

In the example of FIG. 16, information that “Mr. A's gait tends to be a “staggering gait”” is displayed on the screen of the terminal device 180 according to the gait situation determined regarding the user. In the example of FIG. 16, action recommendation information including an instruction policy of “Please make an instruction on how to walk” is displayed on the screen of the terminal device 180 according to the gait situation determined regarding the user. The action recommendation information is optimized in accordance with the instruction policy of the physical therapist. The action recommendation information includes information for urging the physical therapist to make a decision. The physical therapist who has confirmed the information displayed on the screen of the terminal device 180 can determine the instruction policy for the gait of the user by taking action according to the action recommendation information.

As described above, the gait measurement system of the present example embodiment includes the measurement device and the determination device. The measurement device is installed at the user's footwear. The measurement device includes a sensor that measures acceleration and angular velocity. The measurement device generates sensor data using the acceleration and the angular velocity measured by the sensor. The measurement device transmits the generated sensor data to the determination device.

The determination device includes a data acquisition unit, a calculation unit, a storage unit, a determination unit, and an output unit. The data acquisition unit acquires sensor data measured according to the motion of the foot. The calculation unit calculates a relative change value indicating a relative change from the traveling axis for each gait cycle using the acquired time-series data of the sensor data. The calculation unit extracts end points set at the start point and the terminal point of the gait cycle from the time-series data of the sensor data. The calculation unit sets, as a start point of one gait cycle, an end point that precedes in time series among two successive end points. The calculation unit sets, as a terminal point of one gait cycle, an end point following in time series among two successive end points. The calculation unit calculates a relative change value indicating a relative change from the traveling axis at the start point. Specifically, the calculation unit calculates, as relative change values, a relative angle corresponding to an angle formed by a straight line passing through the start point and the terminal point and the traveling axis at the start point on a horizontal plane, and a relative displacement corresponding to a distance between the traveling axis at the start point and the terminal point on the horizontal plane. The storage unit stores the sensor data acquired by the data acquisition unit. The storage unit stores the relative angle and the relative displacement calculated by the calculation unit. The storage unit stores the gait situation determined by the determination unit. The determination unit determines the gait situation using the time-series data of the relative change value in the target period. The output unit outputs gait information including the determined gait situation.

In the present example embodiment, the gait situation is determined using the time-series data of the relative change value including the relative angle and the relative displacement on the horizontal plane calculated for each gait cycle. Using the time-series data of the relative change value on the horizontal plane, it is possible to detect a change in the lateral direction according to the gait situation that cannot be detected by the sensor data. Therefore, when the time-series data of the relative change value in the horizontal plane is used, the gait situation can be determined even for a subject who shows a decline in muscle strength, such as an elderly person or a rehabilitation patient. That is, according to the present example embodiment, the gait situation can be determined for an any subject.

In an aspect of the present example embodiment, the determination unit determines the gait situation using a machine learning model that outputs the gait situation according to the input of the time-series data of the relative change value. According to the present aspect, the gait situation can be determined using the machine learning model.

The determination device according to an aspect of the present example embodiment further includes the information generation unit. The information generation unit generates action recommendation information that is optimized in accordance with the gait situation of the user and urges the user to make a decision using the sensor data associated with the gait situation. The output unit displays gait information including action recommendation information on a screen of a mobile terminal used by the user. According to the present aspect, by presenting the action recommendation information optimized in accordance with the gait situation to the user, it is possible to urge the user to make a decision.

### Second Example Embodiment

Next, a determination device according to a second example embodiment will be described with reference to the drawings. The determination device according to the present example embodiment is different from the determination device according to the first example embodiment in that a relative change value from a start point to a terminal point on the sagittal plane is calculated. The determination device in the present example embodiment constitutes a gait measurement system in combination with the measurement device in the first example embodiment. Hereinafter, the description of the configuration similar to that of the first example embodiment may be omitted. The method of the present example embodiment may be combined with the method of the first example embodiment.

### Configuration

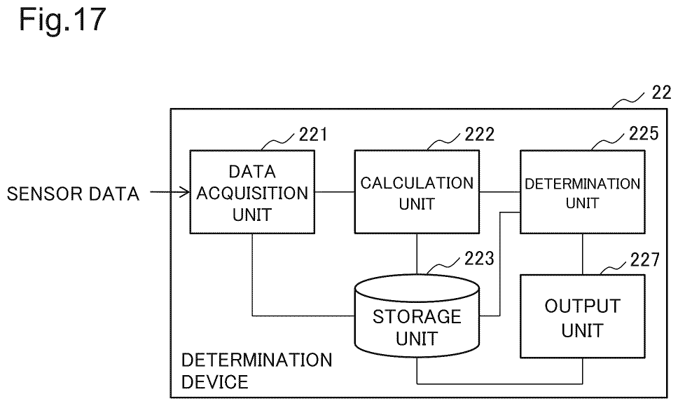

FIG. 17 is a block diagram illustrating an example of a configuration of the determination device in the present disclosure; A determination device 22 includes a data acquisition unit 221, a calculation unit 222, a storage unit 223, a determination unit 225, and an output unit 227.

The data acquisition unit 221 has a configuration similar to that of the data acquisition unit 121 of the first example embodiment. The data acquisition unit 221 acquires time-series data of sensor data from the measurement device.

The calculation unit 222 has a configuration similar to that of the calculation unit 122 of the first example embodiment. The calculation unit 222 extracts an end point of the gait cycle from the time-series data of the sensor data. A section between the successive end points corresponds to one gait cycle. Among two successive end points, the previous end point is set as a start point of one gait cycle. Among two successive end points, the later end point is set as a terminal point of one gait cycle. The calculation unit 222 extracts time-series data of sensor data between two successive end points as a gait waveform for one gait cycle. The calculation unit 222 may normalize the extracted gait waveform.

The calculation unit 222 calculates a relative change value indicating a relative change from the traveling axis on the horizontal plane for each gait cycle using the time-series data (gait waveform) of the sensor data. Specifically, the calculation unit 222 calculates a relative change value from a start point to a terminal point on the sagittal plane (YZ plane) using the extracted gait waveform. The relative change value on the sagittal plane (YZ plane) indicates a relative change from the traveling axis at the start point.

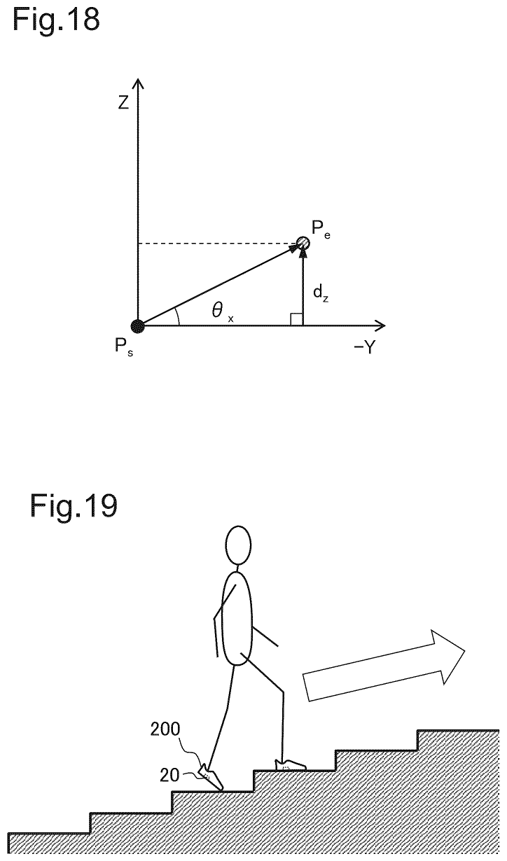

FIG. 18 is a conceptual diagram for describing a relative change value calculated by the determination device in the present disclosure; FIG. 18 illustrates a relative change value of one gait cycle (one stride) from the start point Pₛ to the terminal point Pₑ. The relative change value includes a relative displacement d_{z} and a relative angle θₓ. The relative displacement d_{z} corresponds to an angle formed by the traveling axis (−Y) at the start point Pₛ and a straight line passing through the start point and the terminal point on the sagittal plane (YZ plane). The relative displacement d_{z} corresponds to a distance on the sagittal plane (YZ plane) between the traveling axis (−Y) at the start point Pₛ and the terminal point Pₑ. The calculation unit 222 calculates a relative angle θₓ formed by the traveling axis at the start point and a straight line passing through the start point and the terminal point on the sagittal plane. The calculation unit 222 calculates the relative angle θₓ in the vertical direction from the start point to the terminal point. The calculation unit 222 stores the calculated relative displacement d_{z} and relative angle θₓ in the storage unit 223.

The storage unit 223 has a configuration similar to that of the storage unit 123 of the first example embodiment. The storage unit 223 stores the sensor data acquired by the data acquisition unit 221. The storage unit 223 stores the relative displacement d_{z} and the relative angle θₓ calculated by the calculation unit 222. As will be described later, the storage unit 223 stores the gait situation determined by the determination unit 225.

The determination unit 225 acquires the relative change value in the target period from the storage unit 223. The target period is a time zone over a plurality of gait cycles (stride). The determination unit 225 determines the gait situation in the target period using the time-series data of the relative change value. For example, the determination unit 225 determines the gait situation in the target period using the three-dimensional rotation correction performed using the relative change value. For example, the determination unit 225 determines the gait situation in the target period using a machine learning model (determination model) generated by machine learning. The gait situation includes a gait on a flat ground (normal gait), a gait of going up and down stairs (staircase gait), a gait of going up and down a slope (slope gait), a gait on a road surface with many bumps (uneven gait), and the like. The gait situation is not limited to the example described herein as long as it relates to the gait on a road surface that changes in the vertical direction. The determination unit 225 records the gait situation in the target period in association with the sensor data measured in the gait cycle included in the target period.

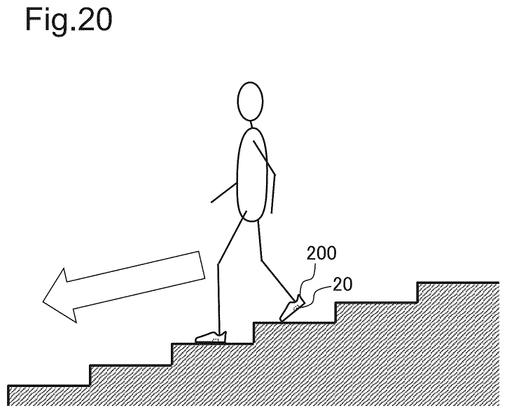

FIGS. 19 to 20 are conceptual diagrams for describing an example of a gait situation to be determined by the determination device in the present disclosure. FIGS. 19 to 20 are views when viewed from the side. FIG. 19 illustrates an example of a gait of going up stairs (staircase gait). FIG. 20 illustrates an example of gait of going down stairs (staircase gait). FIGS. 19 to 20 are examples, and do not limit the gait situation to be determined by the determination device 22. For example, a gait situation such as uneven gait may be included in the determination target by the determination device 22.

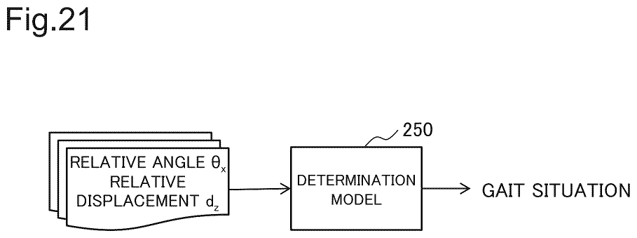

FIG. 21 is a conceptual diagram for describing an example of estimation of a gait situation by the determination device in the present disclosure; A determination model 250 is a machine learning model generated by machine learning. For example, the determination model 250 is a model obtained by learning a data set having the relative angle θₓ and the relative displacement d_{z} as explanatory variables and the gait situation as an objective variable as teacher data. The determination model 250 outputs the gait situation according to the input of the time-series data of the relative angle θₓ and the relative displacement d_{z} calculated using the sensor data measured in the target period. The determination model 250 may be stored in an external storage device constructed in a cloud, a server, or the like. In this case, the determination unit 225 uses the determination model 250 via an interface (not illustrated) connected to the storage device.

For example, the determination model 250 is a learning model trained using a convolutional neural network (CNN) method. For example, the determination model 250 is a model trained using a principal component analysis (PCA) method. For example, the determination model 250 is a learning model trained using a variational autoencoder (VAE). For example, the determination model 250 is a learning model trained using a conditional generative adversarial networks (GAN) method. For example, the determination model 250 may be generated by training using a linear regression algorithm. For example, the determination model 250 may be generated by training using an algorithm of a support vector machine (SVM). For example, the determination model 250 may be generated by training using a Gaussian process regression (GPR) algorithm. For example, the determination model 250 may be generated by training using a random forest (RF) algorithm. The above method is an example, and does not limit the method of training the determination model 250.

The output unit 227 outputs the sensor data stored in the storage unit 223. The sensor data is associated with the gait situation at the measured time point. Information including sensor data associated with a gait situation is also referred to as gait information. For example, the output unit 227 outputs the gait information to a mobile terminal carried by the subject. For example, the output unit 227 outputs the gait information to a terminal device or a server using the sensor data via a mobile terminal carried by the subject. For example, the output unit 227 may be configured to output sensor data to an external system or the like that uses the sensor data.

### Operation

Next, the operation of the determination device 22 will be described with reference to the drawings. The main operation of the determination device 22 is similar to the operation of the determination device 12 of the first example embodiment (FIG. 12). The operation of the determination device 22 is different from the operation of the determination device 12 in the relative change value calculation process in step S12 of FIG. 12. Hereinafter, description of main operations of the determination device 22 will be omitted, and the relative change value calculation process will be described.

### Relative Change Value Calculation Process

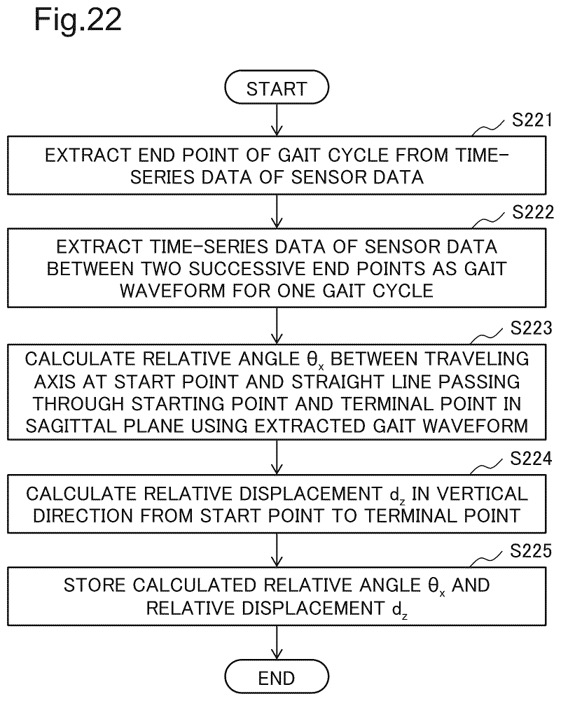

FIG. 22 is a flowchart for describing an example of a relative change value calculation process by the determination device according to the present disclosure; In the description of the processing along the flowchart of FIG. 22, the components of the determination device 22 will be described as the operation subject. The operation subject of the process along the flowchart of FIG. 22 may be the determination device 22.

In FIG. 22, first, the calculation unit 222 extracts an end point of the gait cycle from the time-series data of the sensor data (step S221).

Next, the calculation unit 222 extracts time-series data of sensor data between two successive end points as a gait waveform for one gait cycle (step S222).

Next, using the extracted gait waveform, the calculation unit 222 calculates a relative angle θₓ formed by the traveling axis at the start point and the straight line passing through the start point and the terminal point on the sagittal plane (step S223).

Next, the calculation unit 222 calculates a relative displacement d_{z} in the lateral direction from the start point to the terminal point (step S224). The order of the processing in step S223 and the processing in step S224 may be switched.

Next, the storage unit 223 stores the calculated relative angle θₓ and relative displacement d_{z} (step S225). The relative angle θₓ and the relative displacement d_{z} stored in the storage unit 223 are used for determining the gait situation.

### Application Example

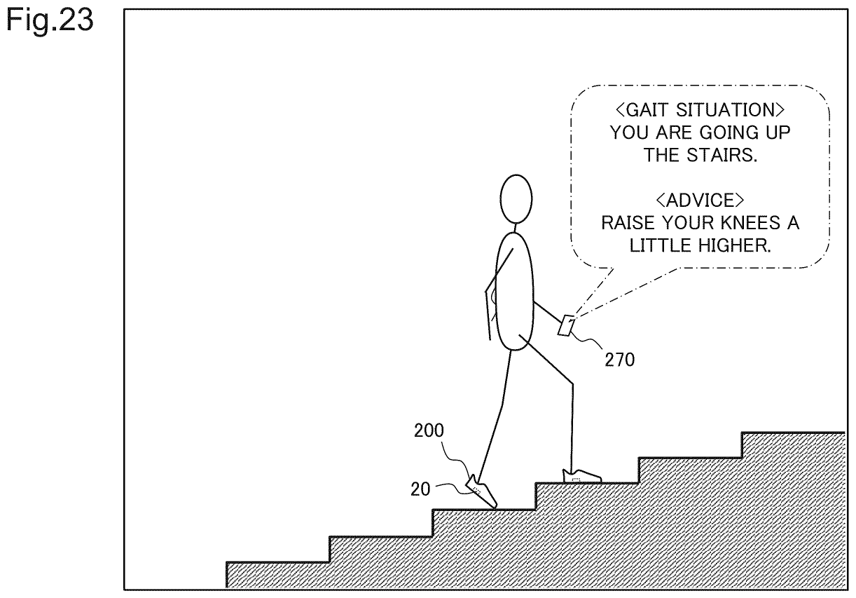

Next, an application example according to the present example embodiment will be described with reference to the drawings. FIGS. 23 to 25 are conceptual diagrams illustrating display examples of information according to the gait situation determined by the determination device in the present disclosure. FIGS. 23 to 25 illustrate an example in which information related to the gait situation of the user is output by voice by a mobile terminal 270 carried by the user who wears the shoes 200 on which a measurement device 20 is disposed and walks. In the example using FIGS. 23 to 25, it is assumed that the information according to the gait situation of the user is generated by an information generation unit (not illustrated) that generates the information according to the gait situation using the sensor data processed by the determination device 22. For example, the information generation unit generates information according to the gait situation of the user using a trained machine learning model constructed in advance.

FIG. 23 illustrates an example in which the gait situation of the user determined by the determination device 22 and the advice according to the gait situation are output by voice by the mobile terminal used by the user. In the example of FIG. 23, information that “You are going up the stairs” is output by voice by the mobile terminal 270 according to the gait situation determined regarding the user. In the example of FIG. 23, action recommendation information that “Raise your knees a little higher” is output by voice by the mobile terminal 270 according to the gait situation determined regarding the user. The action recommendation information is optimized in accordance with the gait situation of the user. The action recommendation information includes information for urging the user to make a decision. The user who has heard the information output by voice by the mobile terminal 270 can take care not to stumble on the stairs by taking action in accordance with the action recommendation information.

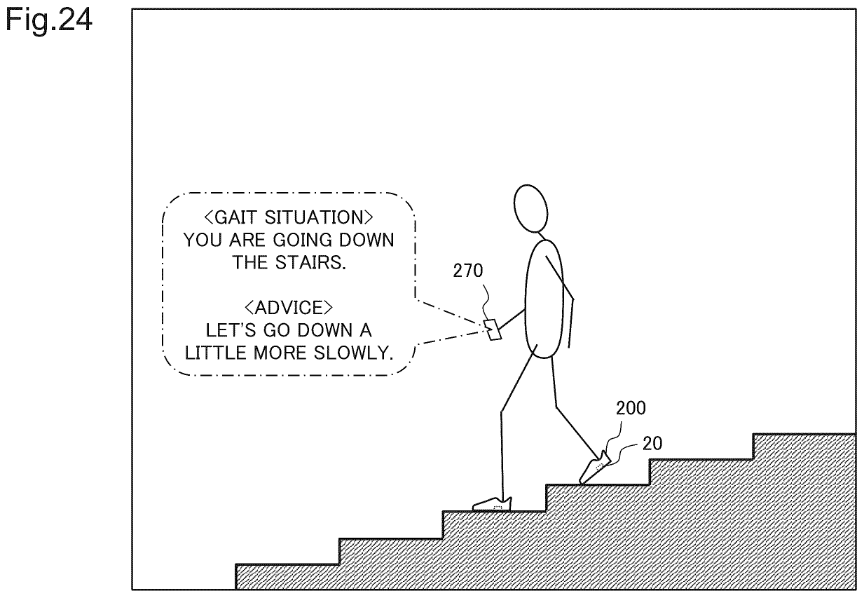

FIG. 24 illustrates an example in which the gait situation of the user determined by the determination device 22 and the advice according to the gait situation are output by voice by the mobile terminal 270 used by the user. In the example of FIG. 24, information that “You are going down the stairs” is output by voice by the mobile terminal 270 according to the gait situation determined regarding the user. In the example of FIG. 24, action recommendation information that “Let's go down a little more slowly” is output by voice by the mobile terminal 270 according to the gait situation determined regarding the user. The action recommendation information is optimized in accordance with the gait situation of the user. The action recommendation information includes information for urging the user to make a decision. The user who has heard the information output by voice by the mobile terminal 270 can take care not to fall down the stairs by taking action according to the action recommendation information.

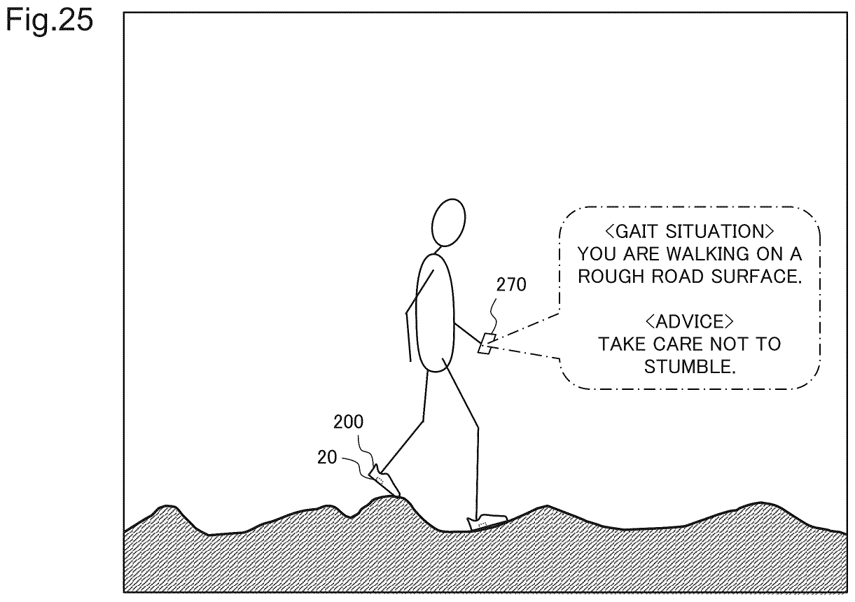

FIG. 25 illustrates an example in which the gait situation of the user determined by the determination device 22 and the advice according to the gait situation are output by voice by the mobile terminal used by the user. In the example of FIG. 25, information that “You are walking on a rough road surface” is output by voice by the mobile terminal 270 according to the gait situation determined regarding the user. In the example of FIG. 25, action recommendation information that “Take care not to stumble” is output by voice by the mobile terminal 270 according to the gait situation determined regarding the user. The action recommendation information is optimized in accordance with the gait situation of the user. The action recommendation information includes information for urging the user to make a decision. The user who has heard the information output by voice by the mobile terminal 270 can take care not to stumble due to unevenness by taking action according to the action recommendation information.

As described above, the determination device of the present example embodiment includes the data acquisition unit, the calculation unit, the storage unit, the determination unit, and the output unit. The data acquisition unit acquires sensor data measured according to the motion of the foot. The calculation unit calculates a relative change value indicating a relative change from the traveling axis for each gait cycle using the acquired time-series data of the sensor data. The calculation unit extracts end points set at the start point and the terminal point of the gait cycle from the time-series data of the sensor data. The calculation unit sets, as a start point of one gait cycle, an end point that precedes in time series among two successive end points. The calculation unit sets, as a terminal point of one gait cycle, an end point following in time series among two successive end points. The calculation unit calculates a relative change value indicating a relative change from the traveling axis at the start point. Specifically, the calculation unit calculates, as relative change values, a relative angle corresponding to an angle formed by a straight line passing through the start point and the terminal point and the traveling axis at the start point on the sagittal plane, and a relative displacement corresponding to a distance between the traveling axis at the start point and the terminal point on the sagittal plane. The storage unit stores the sensor data acquired by the data acquisition unit. The storage unit stores the relative angle and the relative displacement calculated by the calculation unit. The storage unit stores the gait situation determined by the determination unit. The determination unit determines the gait situation using the time-series data of the relative change value in the target period. The output unit outputs gait information including the determined gait situation.

In the present example embodiment, the gait situation is determined using the time-series data of the relative change value including the relative angle and the relative displacement on the sagittal plane calculated for each gait cycle. Using the time-series data of the relative change value on the sagittal plane, it is possible to detect a change in the height direction according to the gait situation that cannot be detected by the sensor data. Therefore, when the time-series data of the relative change value on the sagittal plane is used, the gait situation can be determined even for a subject who shows a decline in muscle strength, such as an elderly person or a rehabilitation patient. That is, according to the present example embodiment, the gait situation can be determined for an any subject.

### Third Example Embodiment

Next, a determination device according to a third example embodiment will be described with reference to the drawings. The determination device according to the present example embodiment is different from that of the first to second example embodiments in that sensor data in a time zone in which the situation is not a normal situation but an exceptional gait situation is removed. The determination device in the present example embodiment constitutes a gait measurement system in combination with the measurement device in the first example embodiment. The method of the present example embodiment may be combined with the methods of the first to second example embodiments. Hereinafter, the description of the configuration similar to that of the first to second example embodiments may be omitted.

### Configuration

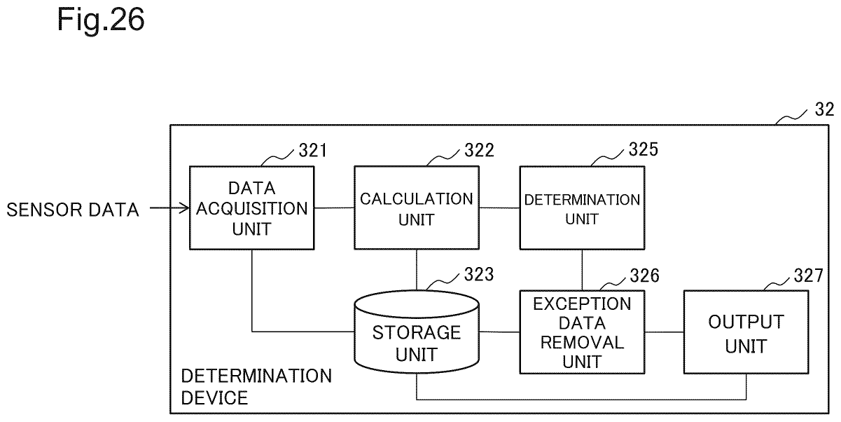

FIG. 26 is a block diagram illustrating an example of a configuration of the determination device in the present disclosure; A determination device 32 includes a data acquisition unit 321, a calculation unit 322, a storage unit 323, a determination unit 325, an exception data removal unit 326, and an output unit 327.

The data acquisition unit 321 has a configuration similar to that of the data acquisition unit 321 of the first example embodiment. The data acquisition unit 321 acquires time-series data of sensor data from the measurement device.

The calculation unit 322 has a configuration similar to that of the calculation unit 322 of the first example embodiment. The calculation unit 322 extracts an end point of the gait cycle from the time-series data of the sensor data. A section between the successive end points corresponds to one gait cycle. Among two successive end points, the previous end point is set as a start point of one gait cycle. Among two successive end points, the later end point is set as a terminal point of one gait cycle. The calculation unit 322 extracts time-series data of sensor data between two successive end points as a gait waveform for one gait cycle. The calculation unit 322 may normalize the extracted gait waveform.

The calculation unit 322 calculates a relative change value indicating a relative change from the traveling axis on the horizontal plane for each gait cycle using the time-series data (gait waveform) of the sensor data. The calculation unit 322 calculates a relative change value from the start point to the terminal point using the extracted gait waveform. For example, the calculation unit 322 calculates a relative change value from a start point to a terminal point on the horizontal plane (XY plane) using the extracted gait waveform. The relative change value on the horizontal plane (XY plane) indicates a relative change from the traveling axis at the start point. For example, the calculation unit 322 calculates a relative change value from a start point to a terminal point on the sagittal plane (YZ plane) using the extracted gait waveform. The relative change value on the sagittal plane (YZ plane) indicates a relative change from the traveling axis at the start point.

The storage unit 323 has a configuration similar to that of the storage unit 323 of the first example embodiment. The storage unit 323 stores the relative change value calculated by the calculation unit 322. For example, the storage unit 323 stores a relative displacement dₓ and a relative angle θ_{z}. For example, the storage unit 323 stores the relative displacement d_{z} and the relative angle θₓ. As will be described later, the storage unit 323 stores the gait situation determined by the determination unit 325. The storage unit 323 stores the sensor data acquired by the data acquisition unit 321.

The determination unit 325 acquires the relative change value in the target period from the storage unit 323. The target period is a time zone over a plurality of gait cycles (stride). The determination unit 325 determines the gait situation in the target period using the time-series data of the relative change value. For example, the determination unit 325 determines the gait situation in the target period using the three-dimensional rotation correction performed using the relative change value. For example, the determination unit 325 determines the gait situation in the target period using a machine learning model (determination model) generated by machine learning.

In a case where the relative change value does not exceed the determination reference value, the determination unit 325 determines that the gait situation is a normal gait. On the other hand, in a case where the relative change value exceeds the determination reference value, the determination unit 325 determines that the gait situation is the exceptional gait. The determination reference value is set for each of the relative displacement dₓ and the relative angle θ_{z} on the horizontal plane, and the relative displacement d_{z} and the relative angle θₓ on the sagittal plane.

The exception data removal unit 326 removes sensor data measured in the target period in which the gait situation is determined to be the exceptional gait different from a normal gait. That is, the exception data removal unit 326 removes the sensor data in the time zone in which the gait situation is determined to be the exceptional gait. On the other hand, the exception data removal unit 326 records the sensor data in the time zone in which the gait situation is determined to be the normal gait in the storage unit 323 in association with the gait situation in the target period.

The output unit 327 outputs the sensor data stored in the storage unit 323. The sensor data is sensor data in a time zone in which the gait situation is determined to be the normal gait. The sensor data is associated with the gait situation at the measured time point. Information including sensor data associated with a gait situation is also referred to as gait information. For example, the output unit 327 outputs the gait information to a mobile terminal carried by the subject. For example, the output unit 327 outputs the gait information to a terminal device or a server using the sensor data via a mobile terminal carried by the subject. For example, the output unit 327 may be configured to output sensor data to an external system or the like that uses the sensor data.

### Operation

Next, the operation of the determination device 32 will be described with reference to the drawings. The main operation of the determination device 32 is similar to the operation of the determination device 12 of the first example embodiment (FIG. 12). The operation of the determination device 32 is different from the operation of the determination device 12 in the processing between step S13 and step S16 in FIG. 12. Hereinafter, description of main operations of the determination device 32 will be omitted, and the relative change value calculation process will be described.

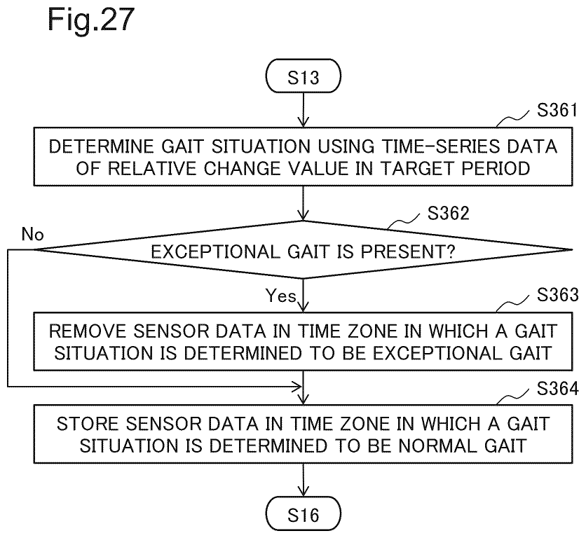

FIG. 27 is a flowchart for describing an example of the operation of the determination device according to the present disclosure; The process of FIG. 27 is inserted between step S13 and step S16 in the flowchart of FIG. 12. In the description of the processing along the flowchart of FIG. 27, the components of the determination device 32 will be described as the operation subject. The operation subject of the process along the flowchart of FIG. 27 may be the determination device 32.

In FIG. 27, after step S13 in FIG. 12, the determination unit 325 determines the gait situation using the time-series data of the relative change value in the target period (step S361).

In a case where the exceptional gait is determined in the target period (Yes in step S362), the exception data removal unit 326 removes the sensor data in the time zone in which the gait situation is determined to be the exceptional gait (step S363).

In a case where exceptional gait is not determined in the target period (No in step S362), or after step S363, the storage unit 323 stores sensor data of a time zone in which the gait situation is determined to be the normal gait (step S364). After step S364, the process proceeds to step S16 in FIG. 12.

As described above, the determination device of the present example embodiment includes the data acquisition unit, the calculation unit, the storage unit, the determination unit, the exception data removal unit, and the output unit. The data acquisition unit acquires sensor data measured according to the motion of the foot. The calculation unit calculates a relative change value indicating a relative change from the traveling axis for each gait cycle using the acquired time-series data of the sensor data. The calculation unit extracts end points set at the start point and the terminal point of the gait cycle from the time-series data of the sensor data. The calculation unit sets, as a start point of one gait cycle, an end point that precedes in time series among two successive end points. The calculation unit sets, as a terminal point of one gait cycle, an end point following in time series among two successive end points. The calculation unit calculates a relative change value indicating a relative change from the traveling axis at the start point. The storage unit stores the sensor data acquired by the data acquisition unit. The storage unit stores the relative angle and the relative displacement calculated by the calculation unit. The storage unit stores the gait situation determined by the determination unit. The determination unit determines the gait situation using the time-series data of the relative change value in the target period. In a case where the relative change value does not exceed the determination reference value, the determination unit determines that the gait situation is a normal gait. On the other hand, in a case where the relative change value exceeds the determination reference value, the determination unit determines that the gait situation is the exceptional gait. The exception data removal unit removes sensor data measured in the target period in which the gait situation is determined to be the exceptional gait different from a normal gait. The exception data removal unit removes the sensor data in the time zone in which the gait situation is determined to be the exceptional gait. On the other hand, the exception data removal unit associates the sensor data in the time zone in which the gait situation is determined to be the normal gait with the gait situation. The output unit outputs gait information including the determined gait situation.

In the present example embodiment, the sensor data in the time zone in which the gait situation is determined to be the exceptional gait is removed, and the sensor data in the time zone in which the gait situation is determined to be the normal gait is associated with the gait situation. According to the present example embodiment, a physical condition or the like can be accurately estimated using only sensor data measured in a normal gait situation.

### Fourth Example Embodiment

Next, a determination device according to a fourth example embodiment will be described with reference to the drawings. The determination device in the present example embodiment is different from that of the first to third example embodiments in that a tag indicating a gait type input by a user is set. The determination device in the present example embodiment constitutes a gait measurement system in combination with the measurement device in the first example embodiment. The method of the present example embodiment may be combined with the methods of the first to third example embodiments. Hereinafter, the description of the configuration similar to that of the first to third example embodiments may be omitted.

### Configuration

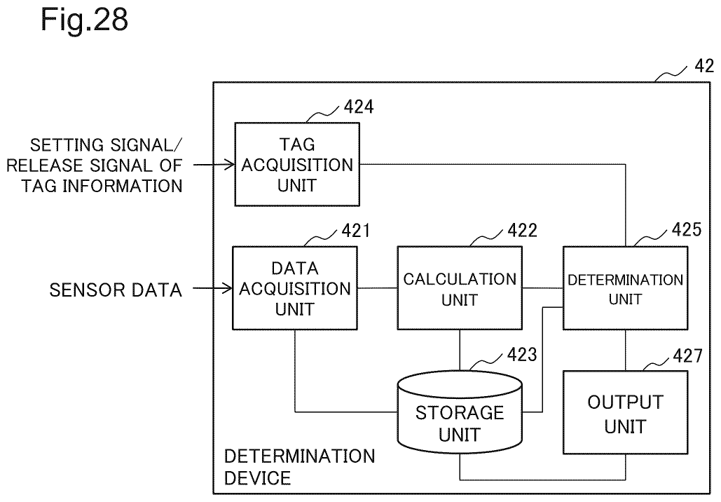

FIG. 28 is a block diagram illustrating an example of a configuration of the determination device in the present disclosure; A determination device 42 includes a data acquisition unit 421, a calculation unit 422, a storage unit 423, a tag acquisition unit 424, a determination unit 425, and an output unit 427.

The data acquisition unit 421 has a configuration similar to that of the data acquisition unit 421 of the first example embodiment. The data acquisition unit 421 acquires time-series data of sensor data from the measurement device.

The calculation unit 422 has a configuration similar to that of the calculation unit 422 of the first example embodiment. The calculation unit 422 extracts an end point of the gait cycle from the time-series data of the sensor data. A section between the successive end points corresponds to one gait cycle. Among two successive end points, the previous end point is set as a start point of one gait cycle. Among two successive end points, the later end point is set as a terminal point of one gait cycle. The calculation unit 422 extracts time-series data of sensor data between two successive end points as a gait waveform for one gait cycle. The calculation unit 422 may normalize the extracted gait waveform.

The calculation unit 422 calculates a relative change value indicating a relative change from the traveling axis on the horizontal plane for each gait cycle using the time-series data (gait waveform) of the sensor data. The calculation unit 422 calculates a relative change value from the start point to the terminal point using the extracted gait waveform. For example, the calculation unit 422 calculates a relative change value from a start point to a terminal point on the horizontal plane (XY plane) using the extracted gait waveform. The relative change value on the horizontal plane (XY plane) indicates a relative change from the traveling axis at the start point. For example, the calculation unit 422 calculates a relative change value from a start point to a terminal point on the sagittal plane (YZ plane) using the extracted gait waveform. The relative change value on the sagittal plane (YZ plane) indicates a relative change from the traveling axis at the start point.

The storage unit 423 has a configuration similar to that of the storage unit 423 of the first example embodiment. The storage unit 423 stores the relative change value calculated by the calculation unit 422. For example, the storage unit 423 stores a relative displacement dₓ and a relative angle θ_{z}. For example, the storage unit 423 stores the relative displacement d_{z} and the relative angle θₓ. As will be described later, the storage unit 423 stores the gait situation determined by the determination unit 425. The storage unit 423 stores the sensor data acquired by the data acquisition unit 421.

The tag acquisition unit 424 acquires a setting signal of a tag indicating the gait type selected by the user. The gait type is obtained by classifying characteristic gait. For example, the gait type includes a staircase gait, a mountain climbing gait, a meandering road gait, an umbrella gait, a snowy road gait, and the like. The tag acquisition unit 424 sets the acquired tag in the determination unit 425. The tag acquisition unit 424 acquires a tag release signal. The tag acquisition unit 424 releases the tag set in the determination unit 425 according to the acquired release signal. For example, the tag is set via a user interface displayed on a screen of a mobile terminal used by the user.

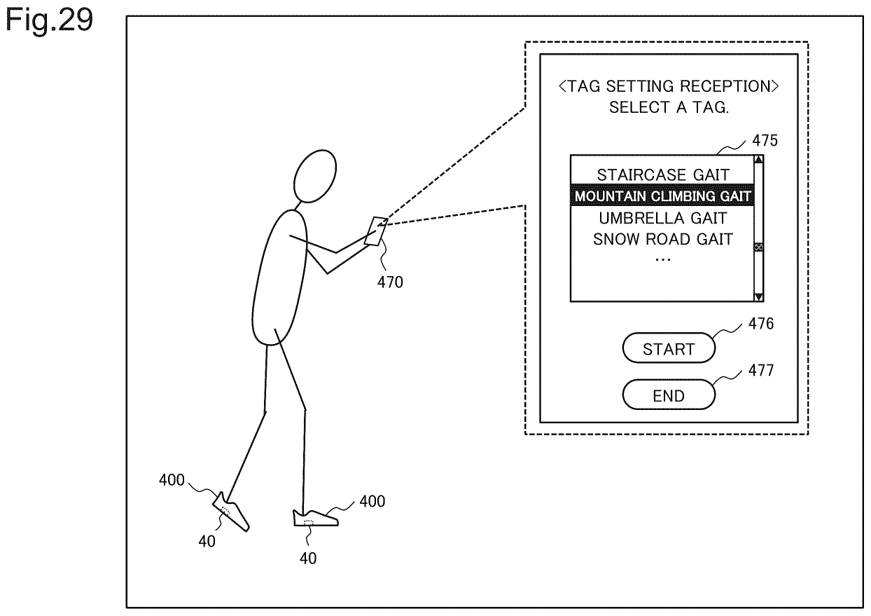

FIG. 29 is a conceptual diagram illustrating an example in which a user interface for inputting a tag in the present disclosure is displayed on a screen of a mobile terminal; The user is gait while wearing shoes 400 on which a measurement device 40 is mounted. On the screen of a mobile terminal 470, a selection area 475 for receiving tag selection is displayed. In the selection area 475, a plurality of gait situation candidates is displayed. In the example of FIG. 29, gait types such as a staircase gait, a mountain climbing gait, a meandering road gait, an umbrella gait, and a snowy road gait are displayed. For example, in the example of FIG. 29, the mountain climbing gait is selected as the gait situation. When the user taps the start button 476, a setting signal of the selected tag is transmitted to the determination device 42, and a tag indicating a gait situation such as the mountain climbing gait is set. When the user taps an end button 477, a tag release signal is transmitted to the determination device 42, and the tag indicating the gait situation such as mountain climbing gait is released.

The determination unit 425 acquires the relative change value in the target period from the storage unit 423. The target period is a time zone over a plurality of gait cycles (stride). The determination unit 425 determines the gait situation in the target period using the time-series data of the relative change value. For example, the determination unit 425 determines the gait situation in the target period using the three-dimensional rotation correction performed using the relative change value. For example, the determination unit 425 determines the gait situation in the target period using a machine learning model (determination model) generated by machine learning. The determination unit 425 records the gait situation in the target period in association with the sensor data measured in the gait cycle included in the target period.

The determination unit 425 receives setting/release of the tag. The determination unit 425 associates the gait situation indicated by the tag with the sensor data measured in the period in which the tag is set. For example, the determination unit 425 preferentially associates the gait situation indicated by the tag with the sensor data measured in the period in which the tag is set. For example, the determination unit 425 may be configured to remove sensor data measured during a period in which a tag is set. In this case, sensor data measured during the period in which the tag is set is not output by the output unit 427. When the tag is released, the determination unit 425 determines the gait situation in the target period using the time-series data of the relative change value.

The output unit 427 outputs the sensor data stored in the storage unit 423. The sensor data is associated with the gait situation at the measured time point. Information including sensor data associated with a gait situation is also referred to as gait information. For example, the output unit 427 outputs the gait information to a mobile terminal carried by the subject. For example, the output unit 427 outputs the gait information to a terminal device or a server using the sensor data via a mobile terminal carried by the subject. For example, the output unit 427 may be configured to output sensor data to an external system or the like that uses the sensor data.

### Operation

Next, the operation of the determination device 42 will be described with reference to the drawings. The main operation of the determination device 42 is similar to the operation of the determination device 12 of the first example embodiment (FIG. 12). The operation of the determination device 42 is different from the operation of the determination device 12 in the processing between step S13 and step S16 in FIG. 12. Hereinafter, description of main operations of the determination device 42 will be omitted, and the relative change value calculation process will be described.

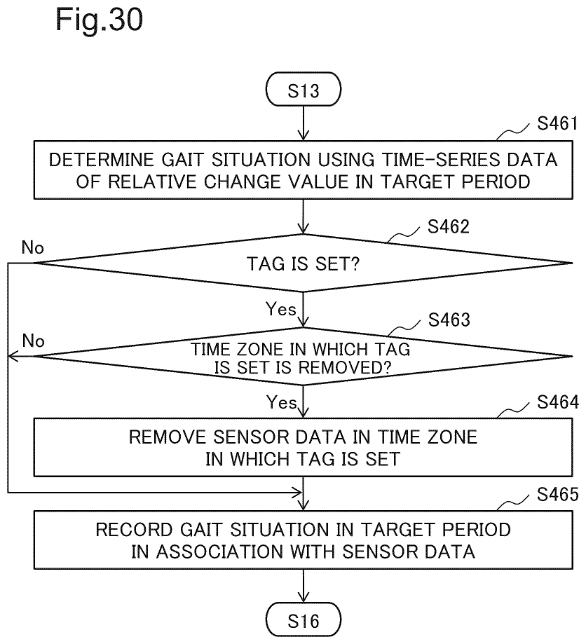

FIG. 30 is a flowchart for describing an example of the operation of the determination device according to the present disclosure; The process of FIG. 30 is inserted between step S13 and step S16 in the flowchart of FIG. 12. In the description of the processing along the flowchart of FIG. 30, the components of the determination device 42 will be described as the operation subject. The operation subject of the process along the flowchart of FIG. 30 may be the determination device 42.

In FIG. 30, after step S13 in FIG. 12, the determination unit 425 determines the gait situation using the time-series data of the relative change value in the target period (step S461).

In a case where the tag is set (Yes in step S462) and the sensor data in the time zone in which the tag is set is removed (Yes in step S463), the determination unit 425 removes the sensor data in the time zone in which the tag is set (step S464). In a case where the tag is set (Yes in step S462) and the sensor data in the time zone in which the tag is set is not removed (No in step S463), the process proceeds to step S465.

In a case of No in steps S462 and S463, or in a case where no tag is set (No in step S462), the storage unit 423 records the gait situation in the target period in association with the sensor data (step S465).

### Application Example

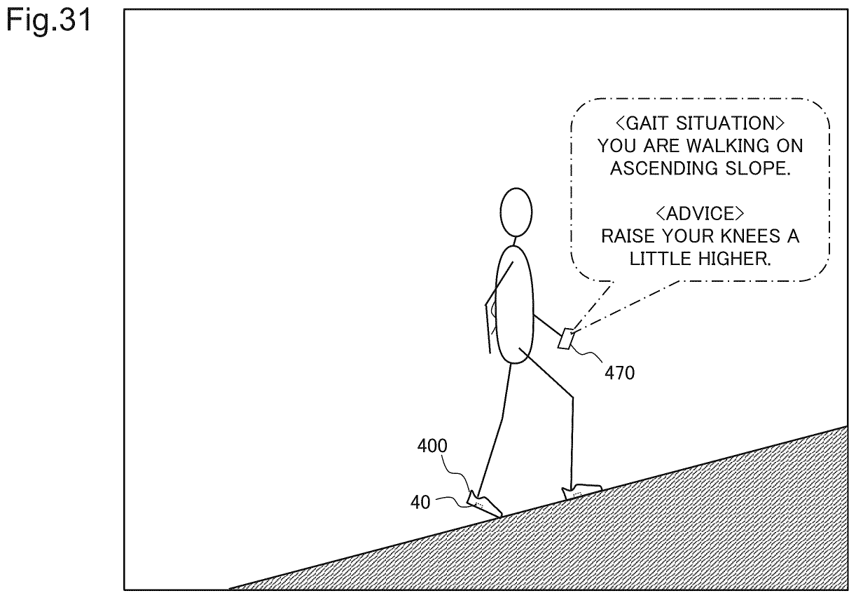

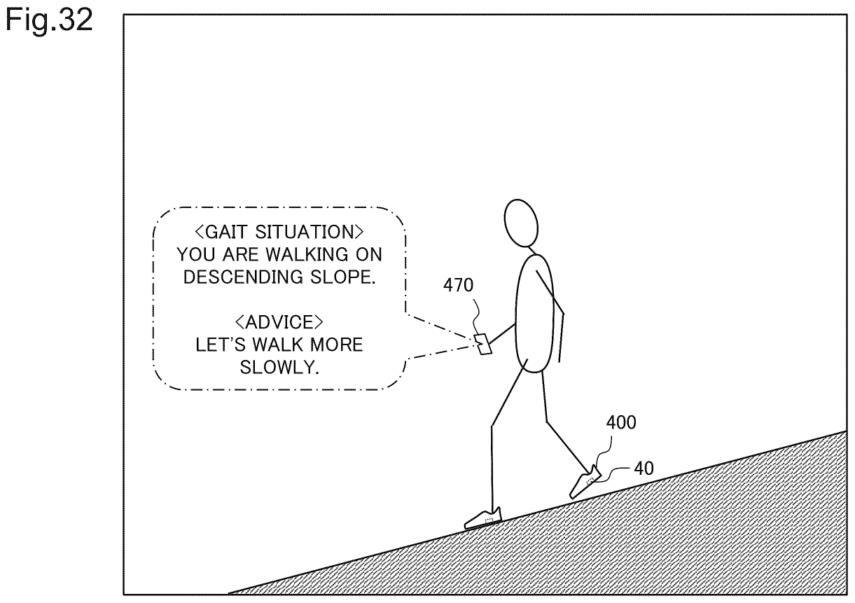

Next, an application example according to the present example embodiment will be described with reference to the drawings. FIGS. 31 to 32 are conceptual diagrams illustrating display examples of information according to the gait situation determined by the determination device in the present disclosure. FIGS. 31 to 32 illustrate an example in which information related to the gait situation is output by voice related to the tag set by the user on the screen of the mobile terminal 470 carried by the user who wears the shoes 400 on which the measurement device 40 is disposed and walks. In the example using FIGS. 31 to 32, it is assumed that the tag of “mountain climbing gait” is selected. In the example using FIGS. 31 to 32, it is assumed that information related to the gait situation of the user is generated by an information generation unit (not illustrated) that generates information using the sensor data processed by the determination device 42. For example, the information generation unit generates information according to the gait situation of the user using a trained machine learning model constructed in advance.

FIG. 31 illustrates an example in which the information related to the gait situation related to the tag set by the user is output by voice by the mobile terminal used by the user. In the example of FIG. 31, information that “You are walking on the ascending slope” is output by voice by the mobile terminal 470 according to the gait situation determined regarding the user. In the example of FIG. 31, action recommendation information that “Raise your knees a little higher” is output by voice by the mobile terminal 470 according to the gait situation determined regarding the user. The action recommendation information is optimized in accordance with the gait situation of the user. The action recommendation information includes information for urging the user to make a decision. The user who has heard the information output by voice by the mobile terminal 470 can walk on the ascending slope while being conscious of raising the knee high by taking action according to the action recommendation information.

FIG. 32 illustrates an example in which the information related to the gait situation related to the tag set by the user is output by voice by the mobile terminal used by the user. In the example of FIG. 32, information that “You are walking on the descending slope” is output by voice by the mobile terminal 470 according to the gait situation determined regarding the user. In the example of FIG. 32, action recommendation information that “Let's walk more slowly” is output by voice by the mobile terminal 470 according to the gait situation determined regarding the user. The action recommendation information is optimized in accordance with the gait situation of the user. The action recommendation information includes information for urging the user to make a decision. The user who has heard the information output by voice by the mobile terminal 470 can walk on the descending slope while being conscious of walking slowly by taking action according to the action recommendation information.

As described above, the determination device of the present example embodiment includes the data acquisition unit, the tag acquisition unit, the calculation unit, the storage unit, the determination unit, and the output unit. The data acquisition unit acquires sensor data measured according to the motion of the foot. The tag acquisition unit acquires a setting signal and a release signal of the tag indicating the gait type selected by the user. The calculation unit calculates a relative change value indicating a relative change from the traveling axis for each gait cycle using the acquired time-series data of the sensor data. The calculation unit extracts end points set at the start point and the terminal point of the gait cycle from the time-series data of the sensor data. The calculation unit sets, as a start point of one gait cycle, an end point that precedes in time series among two successive end points. The calculation unit sets, as a terminal point of one gait cycle, an end point following in time series among two successive end points. The calculation unit calculates a relative change value indicating a relative change from the traveling axis at the start point. The storage unit stores the sensor data acquired by the data acquisition unit. The storage unit stores the relative angle and the relative displacement calculated by the calculation unit. The storage unit stores the gait situation determined by the determination unit. The determination unit determines the gait situation using the time-series data of the relative change value in the target period. The determination unit associates the gait situation indicated by the tag with the sensor data measured in the period in which the tag is set. The output unit outputs gait information including the determined gait situation.

In the present example embodiment, the gait situation indicated by the tag indicating the gait type selected by the user is associated with the sensor data. According to the present aspect, the gait situation can be determined based on not only the gait situation determined by the sensor data but also the tag selected by the user. Therefore, according to the present example embodiment, the gait situation can be more accurately determined in a situation where the exceptional gait is continuously performed.

### Fifth Example Embodiment

Next, a determination device according to a fifth example embodiment will be described with reference to the drawings. The determination device of the present example embodiment has a configuration in which the determination devices of the first to fourth example embodiments are simplified. For example, the functions of the components included in the determination device of the present example embodiment are achieved by the functions of the components included in the determination devices of the first to fourth example embodiments.

### Configuration

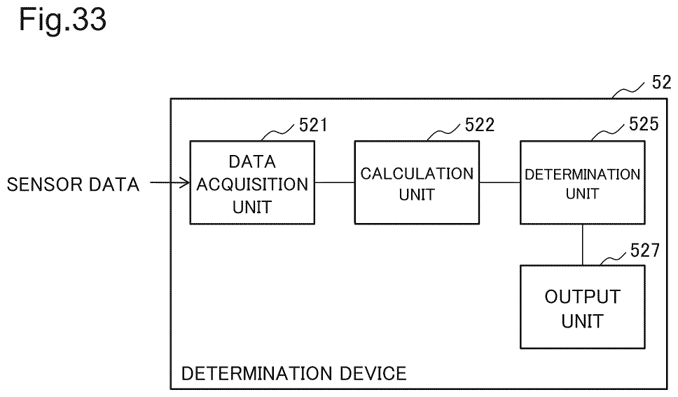

FIG. 33 is a block diagram illustrating an example of a configuration of the determination device in the present disclosure; A determination device 52 includes a data acquisition unit 521, a calculation unit 522, a determination unit 525, and an output unit 527.

The data acquisition unit 521 acquires sensor data measured according to the motion of the foot. The calculation unit 522 calculates a relative change value indicating a relative change from the traveling axis for each gait cycle using the acquired time-series data of the sensor data. The determination unit 525 determines the gait situation using the time-series data of the relative change value in the target period. The output unit 527 outputs gait information including the determined gait situation.

### Operation

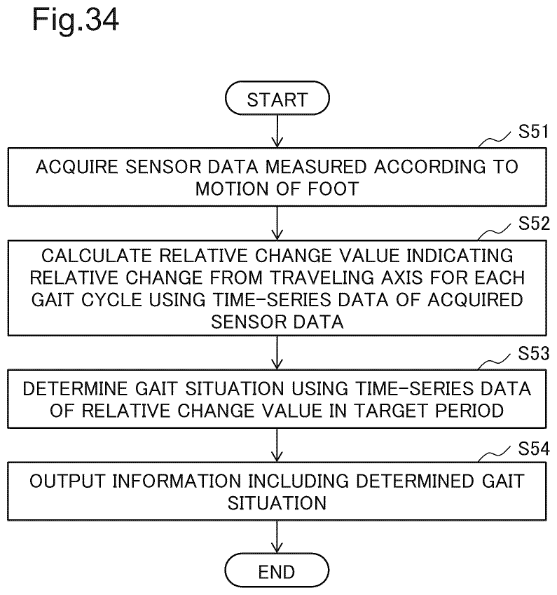

FIG. 34 is a flowchart for describing an example of the operation (determination method) of the determination device according to the present disclosure. In the description of the processing along the flowchart of FIG. 34, the components of the determination device 52 will be described as the operation subject. The operation subject of the process along the flowchart of FIG. 34 may be the determination device 52.

In FIG. 34, first, the data acquisition unit 521 acquires sensor data measured according to the motion of the foot (step S51).

Next, the calculation unit 522 calculates a relative change value indicating a relative change from the traveling axis for each gait cycle using the acquired time-series data of the sensor data (step S52).

Next, the determination unit 525 determines the gait situation using the time-series data of the relative change value in the target period (step S53).

Next, the output unit 527 outputs gait information including the determined gait situation (step S54).

In the present example embodiment, the gait situation is determined using the time-series data of the relative change value calculated for each gait cycle. Using the time-series data of the relative change value, it is possible to detect a change related to the gait situation that cannot be detected by the sensor data. Therefore, when the time-series data of the relative change value is used, the gait situation can be determined even for a subject who shows a decline in muscle strength, such as an elderly person or a rehabilitation patient. That is, according to the present example embodiment, the gait situation can be determined for an any subject.

### Hardware

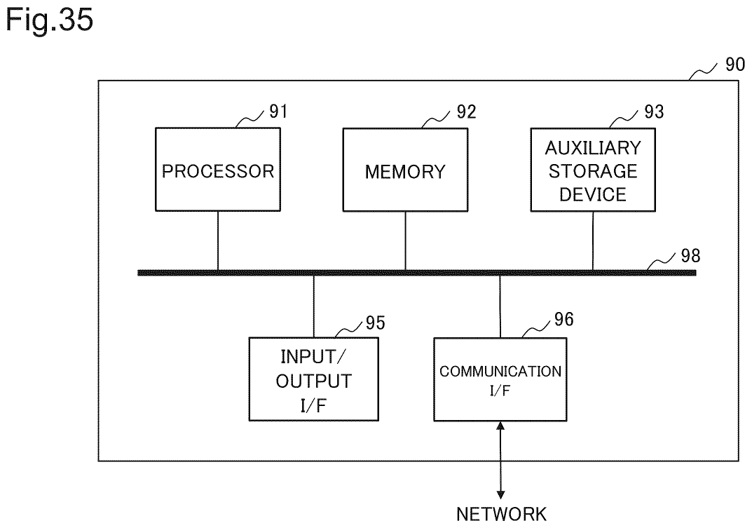

Next, a hardware configuration for executing control and processing in the present disclosure will be described with reference to the drawings. FIG. 35 is a block diagram illustrating an example of a hardware configuration that executes control and processing in the present disclosure. An example of such a hardware configuration is an information processing device 90 (computer). The information processing device 90 is a configuration example for executing control and processing in the present disclosure, and does not limit the scope of the present disclosure.

As illustrated in FIG. 35, the information processing device 90 includes a processor 91, a memory 92, an auxiliary storage device 93, an input/output interface 95, and a communication interface 96. In FIG. 35, the interface is abbreviated as an interface (I/F). The processor 91, the memory 92, the auxiliary storage device 93, the input/output interface 95, and the communication interface 96 are data-communicably connected to each other via a bus 98. The processor 91, the memory 92, the auxiliary storage device 93, and the input/output interface 95 are connected to a network such as the Internet or an intranet via the communication interface 96.

The processor 91 develops a program (instruction) stored in the auxiliary storage device 93 or the like in the memory 92. For example, the program is a software program for executing control and processing in the present disclosure. The processor 91 executes the program developed in the memory 92. The processor 91 executes control and processing in the present disclosure by executing a program.

The memory 92 is a storage device having an area in which a program is developed. A program stored in the auxiliary storage device 93 or the like is developed in the memory 92 by the processor 91. The memory 92 is achieved by, for example, a volatile memory such as a dynamic random access memory (DRAM). A nonvolatile memory such as a magnetoresistive random access memory (MRAM) may be applied as the memory 92.

The auxiliary storage device 93 stores various pieces of data such as programs. For example, the auxiliary storage device 93 is achieved by a local disk such as a hard disk or a flash memory. Various pieces of data may be stored in the memory 92, and the auxiliary storage device 93 may be omitted.

The input/output interface 95 is an interface that connects the information processing device 90 with a peripheral device based on a standard or a specification. The communication interface 96 is an interface that connects to an external system or a device through a network such as the Internet or an intranet in accordance with a standard or a specification. As an interface connected to an external device, the input/output interface 95 and the communication interface 96 may be shared.

An input device such as a keyboard, a mouse, or a touch panel may be connected to the information processing device 90 as necessary. These input devices are used to input of information and settings. In a case where a touch panel is used as the input device, a screen having a touch panel function serves as an interface. The processor 91 and the input device are connected via the input/output interface 95.

The information processing device 90 may be provided with a display device that displays information. In a case where a display device is provided, the information processing device 90 includes a display control device (not illustrated) that controls display of the display device. The information processing device 90 and the display device are connected via the input/output interface 95.

The information processing device 90 may be provided with a drive device. The drive device mediates reading of data and a program stored in a recording medium and writing of a processing result of the information processing device 90 to the recording medium between the processor 91 and the recording medium (program recording medium). The information processing device 90 and the drive device are connected via an input/output interface 95.

The above is an example of a hardware configuration for enabling control and processing in the present disclosure. The hardware configuration of FIG. 35 is an example of a hardware configuration for executing control and processing in the present disclosure, and does not limit the scope of the present disclosure. A program for causing a computer to execute control and processing in the present disclosure is also included in the scope of the present disclosure.

A program recording medium in which a program for executing processing in the present example embodiment is recorded is also included in the scope of the present invention. For example, the program recording medium is a computer-readable non-transitory recording medium. The recording medium can be achieved by, for example, an optical recording medium such as a compact disc (CD) or a digital versatile disc (DVD). The recording medium may be achieved by a semiconductor recording medium such as a universal serial bus (USB) memory or a secure digital (SD) card. The recording medium may be achieved by a magnetic recording medium such as a flexible disk, or another recording medium.

The components in the present disclosure may be combined in any manner. The components in the present disclosure may be implemented by software. The components in the present disclosure may be implemented by a circuit.

The previous description of embodiments is provided to enable a person skilled in the art to make and use the present invention. Moreover, various modifications to these example embodiments will be readily apparent to those skilled in the art, and the generic principles and specific examples defined herein may be applied to other embodiments without the use of inventive faculty. Therefore, the present invention is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

Some or all of the above example embodiments may be described as the following Supplementary Notes, but are not limited to the following.

### Supplementary Note 1

A determination device including
a data acquisition unit that acquires sensor data measured in accordance with a motion of a foot,
a calculation unit that calculates a relative change value indicating a relative change from a traveling axis for each gait cycle using time-series data of the acquired sensor data,
a determination unit that determines a gait situation using time-series data of the relative change value in a target period, and
an output unit that outputs gait information including the determined gait situation.

### Supplementary Note 2

The determination device according to Supplementary Note 1, wherein
the calculation unit
extracts end points set at a start point and a terminal point of a gait cycle from time-series data of the sensor data,
sets, to the start point of one gait cycle, an end point that precedes in time series among the two successive end points,
sets, to the terminal point of one gait cycle, an end point that follows in time series among the two successive end points, and
calculates the relative change value indicating a relative change from a traveling axis at the start point.

### Supplementary Note 3

The determination device according to Supplementary Note 2, wherein
the calculation unit
calculates, as the relative change value, a relative angle corresponding to an angle formed by a straight line passing through the start point and the terminal point and the traveling axis at the start point on a horizontal plane and a relative displacement corresponding to a distance between the traveling axis at the start point and the terminal point on the horizontal plane, and
the determination unit
determines the gait situation using the calculated relative change value.

### Supplementary Note 4

The determination device according to Supplementary Note 2, wherein
the calculation unit
calculates, as the relative change value, a relative angle corresponding to an angle formed by a straight line passing through the start point and the terminal point and the traveling axis at the start point on a sagittal plane and a relative displacement corresponding to a distance between the traveling axis at the start point and the terminal point on the sagittal plane, and
the determination unit
determines the gait situation using the calculated relative change value.

### Supplementary Note 5

The determination device according to any one of Supplementary Notes 1 to 4, further including
an exception data removal unit that removes the sensor data measured in the target period in which the gait situation is determined to be an exceptional gait different from a normal gait, wherein
the determination unit
determines that the gait situation is a normal gait in a case where the relative change value does not exceed a determination reference value, and
determines that the gait situation is an exceptional gait in a case where the relative change value exceeds the determination reference value, and
the exception data removal unit
removes the sensor data in a time zone in which the gait situation is determined to be the exceptional gait, and
associates the sensor data in a time zone in which the gait situation is determined to be the normal gait with the gait situation.

### Supplementary Note 6

The determination device according to any one of Supplementary Notes 1 to 4, further including
a tag acquisition unit that acquires a setting signal and a release signal of a tag indicating a gait type selected by a user, wherein
the determination unit
associates the sensor data measured in a period in which the tag is set with the gait situation indicated by the tag.

### Supplementary Note 7

The determination device according to any one of Supplementary Notes 1 to 4, wherein
the determination unit
determines the gait situation using a machine learning model that outputs the gait situation according to an input of time-series data of the relative change value.

### Supplementary Note 8

The determination device according to any one of Supplementary Notes 1 to 4, further including
an information generation unit that generates, using the sensor data associated with the gait situation, action recommendation information optimized in accordance with the gait situation of a user and urging the user to make a decision, wherein
the output unit
displays the gait information including the action recommendation information on a screen of a mobile terminal used by the user.

### Supplementary Note 9

A determination method executed by a computer, the method including
acquiring sensor data measured in accordance with a motion of a foot,
calculating a relative change value indicating a relative change from a traveling axis for each gait cycle using time-series data of the acquired sensor data,
determining a gait situation using time-series data of the relative change value in a target period, and
outputting gait information including the determined gait situation.

### Supplementary Note 10

A program causes a computer to execute the steps of
acquiring sensor data measured in accordance with a motion of a foot,
calculating a relative change value indicating a relative change from a traveling axis for each gait cycle using time-series data of the acquired sensor data,
determining a gait situation using time-series data of the relative change value in a target period, and
outputting gait information including the determined gait situation.

Some or all of the configurations described in Supplementary Notes 2 to 8 dependent on the above-described Supplementary Note 1 can also be dependent on Supplementary Notes 9 and 10 by the dependency relationship similar to that of Supplementary Notes 2 to 8. Furthermore, some or all of the configurations described as the Supplementary Notes can be similarly dependent on not only the Supplementary Notes 1, 9, and 10, but also various pieces of hardware and software, and various recording means or systems for recording software without departing from the above-described example embodiments.

1. A determination device comprising:
a memory storing instructions; and
a processor connected to the memory and configured to execute the instructions to:
acquire sensor data measured in accordance with a motion of a foot;
calculate a relative change value indicating a relative change from a traveling axis for each gait cycle using time-series data of the acquired sensor data;
determine a gait situation using time-series data of the relative change value in a target period; and
output gait information including the determined gait situation.

2. The determination device according to claim 1, wherein
the processor is configured to execute the instructions to
extract end points set at a start point and a terminal point of a gait cycle from time-series data of the sensor data,
set, to the start point of one gait cycle, an end point that precedes in time series among the two successive end points,
set, to the terminal point of one gait cycle, an end point that follows in time series among the two successive end points, and
calculate the relative change value indicating a relative change from a traveling axis at the start point.

3. The determination device according to claim 2, wherein
the processor is configured to execute the instructions to
calculate, as the relative change value, a relative angle corresponding to an angle formed by a straight line passing through the start point and the terminal point and the traveling axis at the start point on a horizontal plane and a relative displacement corresponding to a distance between the traveling axis at the start point and the terminal point on the horizontal plane, and
determine the gait situation using the calculated relative change value.

4. The determination device according to claim 2, wherein
the processor is configured to execute the instructions to
calculate, as the relative change value, a relative angle corresponding to an angle formed by a straight line passing through the start point and the terminal point and the traveling axis at the start point on a sagittal plane and a relative displacement corresponding to a distance between the traveling axis at the start point and the terminal point on the sagittal plane, and
determine the gait situation using the calculated relative change value.

5. The determination device according to any one of claim 1, wherein
the processor is configured to execute the instructions to
remove the sensor data measured in the target period in which the gait situation is determined to be an exceptional gait different from a normal gait,
determine that the gait situation is a normal gait in a case where the relative change value does not exceed a determination reference value,
determine that the gait situation is an exceptional gait in a case where the relative change value exceeds the determination reference value,
remove the sensor data in a time zone in which the gait situation is determined to be the exceptional gait, and
associate the sensor data in a time zone in which the gait situation is determined to be the normal gait with the gait situation.

6. The determination device according to any one of claim 1, wherein
the processor is configured to execute the instructions to
acquire a setting signal and a release signal of a tag indicating a gait type selected by a user, and
associate the sensor data measured in a period in which the tag is set with the gait situation indicated by the tag.

7. The determination device according to any one of claim 1, wherein
the processor is configured to execute the instructions to
determine the gait situation using a machine learning model that outputs the gait situation according to an input of time-series data of the relative change value.

8. The determination device according to any one of claim 1, wherein
the processor is configured to execute the instructions to
generate, using the sensor data associated with the gait situation, action recommendation information optimized in accordance with the gait situation of a user and urging the user to make a decision,
display the gait information including the action recommendation information on a screen of a mobile terminal used by the user.

9. A determination method executed by a computer, the method comprising:
acquiring sensor data measured in accordance with a motion of a foot;
calculating a relative change value indicating a relative change from a traveling axis for each gait cycle using time-series data of the acquired sensor data;
determining a gait situation using time-series data of the relative change value in a target period; and
outputting gait information including the determined gait situation.

10. A non-transitory recording medium recording a program for causing a computer to execute the steps of:
acquiring sensor data measured in accordance with a motion of a foot;
calculating a relative change value indicating a relative change from a traveling axis for each gait cycle using time-series data of the acquired sensor data;
determining a gait situation using time-series data of the relative change value in a target period; and
outputting gait information including the determined gait situation.
